(19) United States
(12) Patent Application Publication
(10) Pub. No.: US 2025/0122504 A1
(43) Pub. Date: Apr. 17, 2025

(54) COMBINATIONS WITH MODULATORS OF PNPLA3 EXPRESSION

(71) Applicant: AstraZeneca AB, Södertälje (SE)

(72) Inventor: Daniel LINDÉN, Södertälje (SE)

(21) Appl. No.: 18/567,548

(22) PCT Filed: Jun. 7, 2022
(86) PCT No.: PCT/IB2022/055292

§ 371 Date: Dec. 6, 2023

Related U.S. Application Data
(60) Provisional application No. 63/208,299, filed on Jun. 8, 2021.

(51) Int. Cl. C12N 15/113; A61K 38/26; A61P 1/16; A61P 29/00

(52) U.S. Cl. CPC C12N 15/1137; A61K 38/26; A61P 1/16; A61P 29/00; C12N 2310/11; C12N 2310/315; C12N 2310/321; C12N 2310/322; C12N 2310/3341; C12N 2310/341; C12N 2310/351; C12Y 203/01051

(57) ABSTRACT

Provided are methods of treating a liver disease in a subject, comprising administering to the subject: i) an inhibitor of patatin like phospholipase domain containing 3 (PNPLA3) expression; and ii) an agonist of glucagon receptor and/or glucagon-like peptide-1 (GLP-1) receptor. Also provided pharmaceutical and kits comprising i) an inhibitor of PNPLA3 expression; and ii) an agonist of glucagon receptor and/or GLP-1 receptor.

### SEQUENCE LISTING

The instant application contains a Sequence Listing which has been submitted electronically in ASCII format and is hereby incorporated by reference in its entirety. Said ASCII copy, created on Jun. 4, 2021, is named 0098-0076PR1_SL.txt and is 60,642 bytes in size.

### FIELD OF THE DISCLOSURE

The present disclosure provides a method of treating a liver disease in a subject, comprising administering to the subject: i) an inhibitor of patatin like phospholipase domain containing 3 (PNPLA3) expression; and ii) an agonist of glucagon receptor and/or glucagon-like peptide-1 (GLP-1) receptor. Also provided are pharmaceutical and kits comprising i) an inhibitor of PNPLA3 expression; and ii) an agonist of glucagon receptor and/or GLP-1 receptor.

### BACKGROUND

Incidence of liver disease is increasing worldwide, particularly in Western nations. Particularly common is non-alcoholic fatty liver disease (NAFLD). NAFLD covers a spectrum of liver disease from steatosis to nonalcoholic steatohepatitis (NASH) and cirrhosis. NAFLD is defined as fat accumulation in the liver exceeding 5% by weight, in the absence of significant alcohol consumption, steatogenic medication, or hereditary disorders (Kotronen et al, Arterioscler Thromb. Vasc. Biol. 2008, 28:27-38).

Non-alcoholic steatohepatitis (NASH) is NAFLD with signs of inflammation and hepatic injury. NASH is defined histologically by macrovesicular steatosis, hepatocellular ballooning, and lobular inflammatory infiltrates (Sanyal, Hepatol. Res. 2011. 41:670-4). NASH is estimated to affect 2-3% of the general population. In the presence of other pathologies, such as obesity or diabetes, the estimated prevalence increases to 7% and 62% respectively (Hashimoto et al, J. Gastroenterol. 2011. 46 (1): 63-69).

PNPLA3 is a 481 amino acid member of the patatin-like phospholipase domain-containing family that is expressed in the ER and on lipid droplets. In humans, PNPLA3 is highly expressed in the liver, whereas adipose tissue expression is five-fold less (Huang et al, Proc. Natl. Acad. Sci. USA 2010. 107:7892-7).

Glucagon and glucagon-like peptide-1 (GLP-1) derive from pre-proglucagon, a 158 amino acid precursor polypeptide that is differentially proteolytically processed in tissues to form a number of different proglucagon-derived peptides, including glucagon, glucagon-like peptide-1 (GLP-1), glucagon-like peptide-2 (GLP-2) and oxyntomodulin (OXM), that are involved in a wide variety of physiological functions, including glucose homeostasis, insulin secretion, gastric emptying, and intestinal growth, as well as the regulation of food intake. Glucagon is a 29-amino acid peptide that corresponds to amino acids 33 through 61 of proglucagon (53 to 81 of preproglucagon), while GLP-1 is produced as a 37-amino acid peptide that corresponds to amino acids 72 through 108 of proglucagon (92 to 128 of preproglucagon). GLP-1 (7-36) amide or GLP-1 (7-37) acid are biologically active forms of GLP-1, that demonstrate essentially equivalent activity at the GLP-1 receptor.

Glucagon is produced by the endocrine pancreas and activates the glucagon receptor (“GCGR”). Glucagon acts in the liver to raise blood glucose via gluconeogenesis and glycogenolysis. When blood glucose begins to fall, glucagon signals the liver to break down glycogen and release glucose and stimulates production of glucose, causing blood glucose levels to rise toward a normal level. Glucagon has also been shown to increase energy expenditure, increase ketone body production, inhibit lipogenesis and promote fatty acid oxidation, delay gastric emptying and suppress appetite (Müller et al, Proc. Intl. Journal of Molecular Sciences 2020. 21 (2): 383) (Boland et al., Nat Metab., 2020. 2 (5): 413-431).

GLP-1 has different biological activities compared to glucagon. It is secreted from gut L cells and binds to the GLP-1 receptor. Its activities include potentiation of insulin secretion via the incretin effect, inhibition of glucagon secretion, and inhibition of food intake. Both glucagon and GLP-1, acting as agonists at their respective receptors, have been shown to be effective in weight loss. Certain GLP-1 analogs are being sold or are in development for treatment of obesity including, e.g., Liraglutide (VICTOZA® from Novo Nordisk) and Exenatide (Byetta® from AstraZeneca AB).

### SUMMARY OF THE DISCLOSURE

The present disclosure is directed to a method of treating a liver disease in a subject, comprising administering to the subject: i) an inhibitor of patatin like phospholipase domain containing 3 (PNPLA3) expression; and ii) an agonist of glucagon receptor and/or glucagon-like peptide-1 (GLP-1) receptor.

In some embodiments, the inhibitor of PNPLA3 expression is an antisense oligonucleotide that is complementary to a region of a nucleic acid encoding PNPLA3. In some embodiments, the antisense oligonucleotide is complementary to a site within nucleotides 5567-5731 of the nucleic acid encoding PNPLA3. In some embodiments, the antisense oligonucleotide is complementary to a site within nucleotides 5644-5731 of the nucleic acid encoding PNPLA3. In some embodiments, the antisense oligonucleotide is complementary to a site within nucleotides 5567-5642 of the nucleic acid encoding PNPLA3. In some embodiments, the antisense oligonucleotide is complementary to a site within nucleotides 5567-5620 of the nucleic acid encoding PNPLA3. In some embodiments, the nucleic acid encoding PNPLA3 is an mRNA. In some embodiments, the antisense oligonucleotide is from 12 to 30 nucleosides in length. In some embodiments, the antisense oligonucleotide is from 16 to 30 nucleosides in length.

In some embodiments, the antisense oligonucleotide comprises one or more modified sugar moieties. In some embodiments, the one or more modified sugar moieties are 2′-deoxy, 2′-O-methyl, 2′-O-methoxymethyl, 2′-O-methoxyethyl, 2′-fluoro, 4′-CH(CH3)—O-2′, 4′-CH2-O-2′, 4′—(CH2)₂—O-2′ or combinations thereof. In some embodiments, the antisense oligonucleotide comprises one or more modified bases. In some embodiments, the one or more modified bases are 5-methylcytosine. In some embodiments, every cytosine in the antisense oligonucleotide is 5′methylcytosine. In some embodiments, the antisense oligonucleotide comprises one or more non-natural internucleoside linkages. In some embodiments, the one or more internucleoside linkages are phosphorothioate linkages. In some embodiments, every internucleoside linkage is a phosphorothioate linkage.

In some embodiments, the antisense oligonucleotide comprises a sequence having at least 8 contiguous bases of any one of SEQ ID NOs: 2, 3, 4, 5, 6, 7, 8, 9 and 10. In some embodiments, the antisense oligonucleotide comprises one of SEQ ID Nos: 2, 3, 4, 5, 6, 7, 8, 9 and 10. In some embodiments, the antisense oligonucleotide comprises: a) a gap segment consisting of ten linked deoxynucleosides; b) a 5′ wing segment consisting of three linked nucleosides; and c) a 3′ wing segment consisting of three linked nucleosides; wherein the gap segment is positioned between the 5′ wing segment and the 3′ wing segment, wherein each nucleoside of each wing segment comprises a constrained ethyl sugar, wherein each internucleoside linkage is a phosphorothioate linkage, and wherein each cytosine is a 5-methylcytosine.

In some embodiments, the inhibitor of the PNPLA3 expression further comprises a conjugate group.

In some embodiments, the agonist of glucagon receptor and/or GLP-1 receptor is a peptide. In some embodiments, the peptide comprises the amino acid sequence:

(SEQ ID NO: 25)
HX2QGTFTSDX10SX12X13LX15X16X17X18AX20X21FX23X24WL
X27X28GX30

wherein,
(1) X2 is S, X10 is Y, X12 is K, X13 is K, X15 is D, X16 is S, X17 is E, X18 is R, X20 is R, X21 is D, X23 is V, X24 is A, X27 is V, X28 is A, and X30 is G (SEQ ID NO: 14);
(2) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 15);
(3) X2 is S, X10 is K, X12 is K, X13 is Y, X15 is E, X16 is G, X17 is Q, X18 is A, X20 is K, X21 is E, X23 is I, X24 is A, X27 is E, X28 is K, and X30 is R (SEQ ID NO: 20);
(4) X2 is S, X10 is K, X12 is S, X13 is Y, X15 is D, X16 is S, X17 is R, X18 is S, X20 is R, X21 is D, X23 is V, X24 is A, X27 is E, X28 is A, and X30 is G (SEQ ID NO: 18);
(5) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 33); or
(6) X2 is S, X10 is K, X12 is S, X13 is Y, X15 is D, X16 is S, X17 is R, X18 is R, X20 is R, X21 is D, X23 is V, X24 is A, X27 is E, X28 is A, and X30 is G (SEQ ID NO:19).

In some embodiments, the peptide comprises the amino acid sequence

(SEQ ID NO: 33)
HSQGTFTSDKSEYLDSERARDFVAWLEAGG.

In some embodiments, the peptide further comprises a modification to an amino acid in the amino acid sequence. In some embodiments, the modification is the addition of an acyl moiety. In some embodiments, the modification is a palmitoyl moiety on the N (epsilon) group of a lysine residue. In some embodiments, the palmitoyl group is linked to the lysine via a linker. In some embodiments, the linker is gamma glutamic acid. In at least one embodiment, the peptide is HSQGTFTSDKSEYLDSERARDFVAWLEAGG (SEQ ID NO: 33), wherein the lysine is modified with a palmitoyl moiety via a glutamic acid linker.

In some embodiments, the inhibitor of PNPLA3 expression and the agonist of glucagon receptor and/or GLP-1 receptor are administered concomitantly. In some embodiments, the inhibitor of PNPLA3 expression and the agonist of glucagon receptor and/or GLP-1 receptor are administered within 1 hour of one another. In some embodiments, the inhibitor of PNPLA3 expression and the agonist of glucagon receptor and/or GLP-1 receptor are administered within 24 hours of one another. In some embodiments, the inhibitor of PNPLA3 expression and the agonist of glucagon receptor and/or GLP-1 receptor are administered within 72 hours of one another. In some embodiments, the inhibitor of PNPLA3 expression and the agonist of glucagon receptor and/or GLP-1 receptor are administered within one week of one another. In some embodiments, the inhibitor of PNPLA3 expression and the agonist of glucagon receptor and/or GLP-1 receptor are administered within two weeks of one another. In some embodiments, the inhibitor of PNPLA3 expression is administered parenterally. In some embodiments, the inhibitor of PNPLA3 expression is administered daily, twice daily or three times daily. In some embodiments, inhibitor of PNPLA3 expression is administered weekly, twice weekly or three times weekly. In some embodiments, the inhibitor of PNPLA3 expression is administered monthly, twice monthly or three times monthly.

In some embodiments, the agonist of glucagon receptor and/or GLP-1 receptor is administered parenterally. In some embodiments, the agonist of glucagon receptor and/or GLP-1 receptor is administered daily, twice daily or three times daily. In some embodiments, the agonist of glucagon receptor and/or GLP-1 receptor is administered weekly, twice weekly or three times weekly. In some embodiments, the agonist of glucagon receptor and/or GLP-1 receptor is administered monthly, twice monthly or three times monthly.

In some embodiments, the subject is obese and/or has type 2 diabetes mellitus. In some embodiments, the liver disease is non-alcoholic fatty liver disease (NAFLD). In some embodiments, the liver disease is nonalcoholic steatohepatitis (NASH). In some embodiments, the liver disease is liver fibrosis and/or cirrhosis.

In some embodiments, the disclosure is directed to a method of reducing steatosis in the liver of a subject having a liver disease, comprising administering to the subject: i) an inhibitor of patatin like phospholipase domain containing 3 (PNPLA3) expression; and ii) an agonist of glucagon receptor and/or glucagon-like peptide-1 (GLP-1) receptor.

In some embodiments, the total liver steatosis is reduced in the subject compared to total liver steatosis when the inhibitor of PNPLA3 expression or the agonist of glucagon receptor GLP-1 receptor is administered alone. In some embodiments, the total liver steatosis is reduced in the subject at least 30% compared to total liver steatosis when the inhibitor of PNPLA3 expression or the agonist of glucagon receptor GLP-1 receptor is administered alone. In some embodiments, the total liver steatosis is reduced in the subject at least 30% compared to total liver steatosis when the inhibitor of PNPLA3 expression or the agonist of glucagon receptor GLP-1 receptor is administered alone. In some embodiments, the liver disease is non-alcoholic fatty liver disease (NAFLD). In some embodiments, the liver disease is nonalcoholic steatohepatitis. In some embodiments, the liver disease is liver fibrosis.

In some embodiments, the disclosure provides a method of reducing inflammation in the liver of a subject having a nonalcoholic fatty liver disease (NAFLD), comprising administering to the subject: i) an inhibitor of patatin like phospholipase domain containing 3 (PNPLA3) expression; and ii) an agonist of glucagon receptor and/or glucagon-like peptide-1 (GLP-1) receptor. In some embodiments, the inflammation in the liver is reduced in the subject at least 50% compared to inflammation in the liver when the inhibitor of PNPLA3 expression or the agonist of glucagon receptor GLP-1 receptor is administered alone.

In some embodiments, the disclosure provides a method of reducing liver collagen in a subject having a liver disease, comprising administering to the subject: i) an inhibitor of patatin like phospholipase domain containing 3 (PNPLA3) expression; and ii) an agonist of glucagon receptor and/or glucagon-like peptide-1 (GLP-1) receptor. In some embodiments, the liver collagen is reduced in the subject at least 25% compared to liver collagen when the inhibitor of PNPLA3 expression or the agonist of glucagon receptor GLP-1 receptor is administered alone.

In some embodiments, the disclosure provides a pharmaceutically acceptable composition comprising: i) an inhibitor of patatin like phospholipase domain containing 3 (PNPLA3) expression; ii) an agonist of glucagon receptor and/or glucagon-like peptide-1 (GLP-1) receptor; and iii) at least one pharmaceutically acceptable excipient. In some embodiments, the composition is formulated for parenteral administration.

In some embodiments, the disclosure provides a kit comprising: i) an inhibitor of patatin like phospholipase domain containing 3 (PNPLA3) expression; and ii) an agonist of glucagon receptor and/or glucagon-like peptide-1 (GLP-1) receptor.

### BRIEF DESCRIPTION OF THE DRAWINGS

The following drawings form part of the present specification and are included to further demonstrate exemplary embodiments of certain aspects of the present disclosure.

FIG. 1A shows plots of percent body weight change for homozygous Pnpla3^{148M/M} knock-in mice fed a NASH inducing diet for 36 weeks and treated with dosed with either 1) control ASO+saline, 2) Pnpla3 ASO+saline, 3) control ASO+Cotadutide, or 4) Pnpla3 ASO+Cotadutide for 14 weeks as described in Example 1. FIG. 1B shows plots of liver mPnpla3 mRNA concentrations for the same mice, measured as described in Example 1.

FIG. 2 shows plots of total liver steatosis (2A), macrovesicular steatosis (2B) and microvesicular steatosis (2C) measured from stained liver sections taken from the mice described above for FIGS. 1A and 1n Example 1. FIG. 2D shows images of the stained sections, with the percentage of total lipid droplets per area provided for each section.

FIG. 3A shows plots of the percentage of liver macrophages measured for liver sections taken from the mice described above for FIGS. 1A and 1n Example 1. FIG. 3B shows plots of the inflammation scores for livers obtained from the mice described above for FIGS. 1A and 1n Example 1.

FIG. 4 shows plots of the NAFLD activity score (NAS) calculated as described in Example 1, for the mice described above for FIGS. 1A and 1n Example 1.

FIG. 5 shows plots of the percentage of liver collagen A1A in liver sections taken from the mice described above for FIGS. 1A and 1n Example 1. Liver collagen is measured as described in Example 1.

### DETAILED DESCRIPTION OF THE DISCLOSURE

The present disclosure provides a method of treating liver disease, e.g., NASH and or NAFLD. In some embodiments, the disclosure provides a method of treating a liver disease in a subject, comprising administering to the subject: i) an inhibitor of patatin like phospholipase domain containing 3 (PNPLA3) expression; and ii) an agonist of glucagon receptor and/or glucagon-like peptide-1 (GLP-1) receptor.

### Definitions

Unless otherwise indicated, the following terms have the following meanings:

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability, depending on the situation. In embodiments, one of skill in the art will understand the level of variability indicated by the term “about,” due to the context in which it is used herein. It should also be understood that use of the term “about” also includes the specifically recited value.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, “between” is a range inclusive of the ends of the range. For example, a number between x and y explicitly includes the numbers x and y, and any numbers that fall within x and y.

“2′-deoxynucleoside” means a nucleoside comprising 2′-H (H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“2′-O-methoxyethyl” (also 2′-MOE) refers to a 2′-O (CH₂)₂—OCH₃) in the place of the 2′-—OH group of a ribosyl ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

“3′ target site” refers to the nucleotide of a target nucleic acid which is complementary to the 3′-most nucleotide of a particular compound.

“5′ target site” refers to the nucleotide of a target nucleic acid which is complementary to the 5′-most nucleotide of a particular compound.

“5-methylcytosine” means a cytosine with a methyl group attached to the 5 position.

“Administration” or “administering” refers to routes of introducing a compound or composition provided herein to an individual to perform its intended function. An example of a route of administration that can be used includes, but is not limited to parenteral administration, such as subcutaneous, intravenous, or intramuscular injection or infusion.

“Administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.

“Amelioration” refers to an improvement or lessening of at least one indicator, sign, or symptom of an associated disease, disorder, or condition. In certain embodiments, amelioration includes a delay or slowing in the progression or severity of one or more indicators of a condition or disease. The progression or severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense compound” means a compound comprising an oligonucleotide and optionally one or more additional features, such as a conjugate group or terminal group. Examples of antisense compounds include single-stranded and double-stranded compounds, such as, oligonucleotides, ribozymes, siRNAs, shRNAs, ssRNAs, and occupancy-based compounds.

“Antisense oligonucleotide” means an oligonucleotide having a nucleobase sequence that is complementary to a target nucleic acid or region or segment thereof. In certain embodiments, an antisense oligonucleotide is specifically hybridizable to a target nucleic acid or region or segment thereof.

“cEt” or “constrained ethyl” means a ribosyl bicyclic sugar moiety wherein the second ring of the bicyclic sugar is formed via a bridge connecting the 4′-carbon and the 2′-carbon, wherein the bridge has the formula: 4′-CH (CH₃)—O-2′, and wherein the methyl group of the bridge is in the S configuration.

“cEt nucleoside” means a nucleoside comprising a cEt modified sugar moiety.

“Chemical modification” in a compound describes the substitutions or changes through chemical reaction, of any of the units in the compound relative to the original state of such unit. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Conjugate group” means a group of atoms that is attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Dose” means a specified quantity of a compound or pharmaceutical agent provided in a single administration, or in a specified time period. In certain embodiments, a dose may be administered in two or more boluses, tablets, or injections. For example, in certain embodiments, where subcutaneous administration is desired, the desired dose may require a volume not easily accommodated by a single injection. In such embodiments, two or more injections may be used to achieve the desired dose. In certain embodiments, a dose may be administered in two or more injections to minimize injection site reaction in an individual. In other embodiments, the compound or pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“Effective amount” means the amount of compound sufficient to effectuate a desired physiological outcome in an individual in need of the compound. The effective amount may vary among individuals depending on the health and physical condition of the individual to be treated, the taxonomic group of the individuals to be treated, the formulation of the composition, assessment of the individual's medical condition, and other relevant factors.

“Gapmer” means an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Immediately adjacent” means there are no intervening elements between the immediately adjacent elements of the same kind (e.g., no intervening nucleobases between the immediately adjacent nucleobases).

“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“Linker-nucleoside” means a nucleoside that links an oligonucleotide to a conjugate moiety. Linker-nucleosides are located within the conjugate linker of a compound. Linker-nucleosides are not considered part of the oligonucleotide portion of a compound even if they are contiguous with the oligonucleotide.

“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.

“Modulating” refers to changing or adjusting a feature in a cell, tissue, organ or organism. For example, modulating PNPLA3 RNA can mean to increase or decrease the level of PNPLA3 RNA and/or PNPLA3 protein in a cell, tissue, organ or organism. A “modulator” effects the change in the cell, tissue, organ or organism. For example, a PNPLA3 compound can be a modulator that decreases the amount of PNPLA3 RNA and/or PNPLA3 protein in a cell, tissue, organ or organism.

“MOE” means methoxyethyl.

“Non-bicyclic modified sugar” or “non-bicyclic modified sugar moiety” means a modified sugar moiety that comprises a modification, such as a substituent, that does not form a bridge between two atoms of the sugar to form a second ring.

“Oligomeric compound” means a compound comprising a single oligonucleotide and optionally one or more additional features, such as a conjugate group or terminal group.

“Oligonucleotide” means a polymer of linked nucleosides each of which can be modified or unmodified, independent one from another. Unless otherwise indicated, oligonucleotides consist of 8-80 linked nucleosides. “Modified oligonucleotide” means an oligonucleotide, wherein at least one sugar, nucleobase, or internucleoside linkage is modified. “Unmodified oligonucleotide” means an oligonucleotide that does not comprise any sugar, nucleobase, or internucleoside modification.

“Phosphorothioate linkage” means a modified phosphate linkage in which one of the non-bridging oxygen atoms is replaced with a sulfur atom. A phosphorothioate internucleoside linkage is a modified internucleoside linkage.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“RefSeq No.” is a unique combination of letters and numbers assigned to a sequence to indicate the sequence is for a particular target transcript (e.g., target gene). Such sequence and information about the target gene (collectively, the gene record) can be found in a genetic sequence database. Genetic sequence databases include the NCBI Reference Sequence database, GenBank, the European Nucleotide Archive, and the DNA Data Bank of Japan (the latter three forming the International Nucleotide Sequence Database Collaboration or INSDC).

“RNAi compound” means an antisense compound that acts, at least in part, through RISC or Ago₂, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.

“Sugar moiety” means an unmodified sugar moiety or a modified sugar moiety. “Unmodified sugar moiety” or “unmodified sugar” means a 2′-OH (H) ribosyl moiety, as found in RNA (an “unmodified RNA sugar moiety”), or a 2′-H (H) moiety, as found in DNA (an “unmodified DNA sugar moiety”). “Modified sugar moiety” or “modified sugar” means a modified furanosyl sugar moiety or a sugar surrogate. “Modified furanosyl sugar moiety” means a furanosyl sugar comprising a non-hydrogen substituent in place of at least one hydrogen or hydroxyl of an unmodified sugar moiety. In certain embodiments, a modified furanosyl sugar moiety is a 2′-substituted sugar moiety. Such modified furanosyl sugar moieties include bicyclic sugars and non-bicyclic sugars.

“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary compounds or nucleic acids.

“Therapeutically effective amount” means an amount of a compound, pharmaceutical agent, or composition that provides a therapeutic benefit to an individual.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, glycosylation, acetylation, phosphorylation, amidation, derivatization by known protecting/blocking groups, proteolytic cleavage, or modification by non-naturally occurring amino acids.

The term “sequence identity” as used herein refers to a relationship between two or more polynucleotide sequences or between two or more polypeptide sequences. When a position in one sequence is occupied by the same nucleic acid base or amino acid in the corresponding position of the comparator sequence, the sequences are said to be “identical” at that position. The percentage “sequence identity” is calculated by determining the number of positions at which the identical nucleic acid base or amino acid occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90 (12): 5873-5877, 1993). When utilizing “BLAST 2 Sequences,” parameters that were default parameters as of Sep. 1, 2004, can be used for word size (3), open gap penalty (11), extension gap penalty (1), gap drop-off (50), expect value (10), and any other required parameter including but not limited to matrix option.

### PNPLA3

In some embodiments, the disclosure provides a method of treating a liver disease by administering a PNPLA3 inhibitor. PNPLA3 is a 481 amino acid member of the patatin-like phospholipase domain-containing family that is expressed in the ER and on lipid droplets. In humans, PNPLA3 is highly expressed in the liver, whereas adipose tissue expression is five-fold less (Huang et al, Proc. Natl. Acad. Sci. USA 2010. 107:7892-7). In some embodiments, PNPLA3 refers to SEQ ID NO: 1. “PNPLA3” means any nucleic acid or protein of PNPLA3. “PNPLA3 nucleic acid” means any nucleic acid encoding PNPLA3. For example, in certain embodiments, a PNPLA3 nucleic acid includes a DNA sequence encoding PNPLA3, an RNA sequence transcribed from DNA encoding PNPLA3 (including genomic DNA comprising introns and exons), and an mRNA sequence encoding PNPLA3. “PNPLA3 mRNA” means an mRNA encoding a PNPLA3 protein. The target may be referred to in either upper or lower case.

In some embodiments, the disclosure provides methods, compounds and compositions for inhibiting PNPLA3 (PNPLA3) expression for the treatment of liver disease in combination with an agonist of glucagon receptor and/or GLP-1 receptor. Certain embodiments provided herein relate to methods of treating liver disease by administering an inhibitor of PNPLA3. “PNPLA3 specific inhibitor” can refer to any agent capable of specifically inhibiting PNPLA3 RNA and/or PNPLA3 protein expression or activity at the molecular level. For example, PNPLA3 specific inhibitors include nucleic acids (including antisense compounds), peptides, antibodies, small molecules, and other agents capable of inhibiting the expression of PNPLA3 RNA and/or PNPLA3 protein.

The terms “inhibitor of PNPLA3,” “PNPLA3 inhibitor” and “PNPLA3 inhibitor of expression” are used interchangeably herein. Inhibiting PNPLA3 expression can be useful for treating, preventing, or ameliorating a disease associated with PNPLA3 in an individual, by administration of a compound that targets PNPLA3. In certain embodiments, the PNPLA3 inhibitor can be a PNPLA3 specific inhibitor. In certain embodiments, the PNPLA3 inhibitor can be an antisense compound, an oligomeric compound, or an oligonucleotide targeted to PNPLA3. In some embodiments, the PNPLA3 is an antisense oligonucleotide. In some embodiments, the oligonucleotide is an siRNA, microRNA targeting oligonucleotide, or a single-stranded RNAi compound, such as small hairpin RNAs (shRNAs), single-stranded siRNAs (ssRNAs), and microRNA mimics.

In some embodiments, the PNPLA3 inhibitor is an antisense oligonucleotide targeted to a PNPLA3 nucleic acid. In certain embodiments, the PNPLA3 nucleic acid has the sequence set forth in U.S. Pat. No. 10,774,333, incorporated by reference, e.g., RefSeq or GENBANK Accession No. NM_025225.2; NC_000022.11 truncated from nucleotides 43921001 to 43,954,500 (SEQ ID NO: 2); AK123806.1; BQ686328.1; BF762711.1; DA290491.1; and the sequences listed as “SEQ ID Nos 7, 8, 9, and 10” in U.S. Pat. No. 10,774,333. In certain embodiments, the PNPLA3 inhibitor is an antisense oligonucleotide or oligomeric compound. In certain embodiments, the PNPLA3 inhibitor is single-stranded. In certain embodiments, the PNPLA3 inhibitor is double-stranded.

In certain embodiments, the PNPLA3 inhibitor comprises a modified oligonucleotide 16 linked nucleosides in length. In certain embodiments, the PNPLA3 inhibitor is an antisense compound or oligomeric compound.

In some embodiments, the PNPLA3 inhibitor is a modified oligonucleotide 12 to 30 linked nucleosides in length and having a nucleobase sequence comprising any of the nucleobase sequences as described in U.S. Pat. No. 10,774,333, incorporated herein by reference, e.g., any one of “SEQ ID NOs: 17-2169” of U.S. Pat. No. 10,774,333. In certain embodiments, the PNPLA3 inhibitor is an antisense compound or oligomeric compound. In certain embodiments, the PNPLA3 inhibitor is single-stranded. In certain embodiments, the PNPLA3 inhibitor is double-stranded. In certain embodiments, the PNPLA3 inhibitor is a modified oligonucleotide of 16 to 30 linked nucleosides in length.

In some embodiments, the PNPLA3 inhibitor comprises a modified oligonucleotide consisting of any of the nucleobase sequences as found in U.S. Pat. No. 10,774,333, incorporated herein by reference, e.g., any one of “SEQ ID NOs: 17-2169” of U.S. Pat. No. 10,774,333. In certain embodiments, the PNPLA3 inhibitor is an antisense compound or oligomeric compound. In certain embodiments, the PNPLA3 inhibitor is single-stranded. In certain embodiments, the PNPLA3 inhibitor is double-stranded.

In some embodiments, the PNPLA3 inhibitor comprises a modified oligonucleotide 12 to 30 linked nucleosides in length and complementary within nucleobases as found in U.S. Pat. No. 10,774,333, incorporated herein by reference, e.g., nucleobases 5567-5642, 5644-5731, 5567-5731, 5567-5620, 13697-13733, 20553-20676, 20664-20824, 20553-20824, and 25844-25912 of SEQ ID NO: 1 wherein said modified oligonucleotide is at least 85%, at least 90%, at least 95%, or 100% complementary to SEQ ID NO: 1. In certain embodiments, the PNPLA3 inhibitor is an antisense compound or oligomeric compound. In certain embodiments, the PNPLA3 inhibitor is single-stranded. In certain embodiments, the PNPLA3 inhibitor is double-stranded. In certain embodiments, the modified oligonucleotide is 16 to 30 linked nucleosides in length.

In some embodiments, the inhibitor of PNPLA3 expression is an antisense oligonucleotide that is complementary to a region of a nucleic acid encoding PNPLA. In certain embodiments, the PNPLA3 inhibitor target nucleotides 5567-5620 of a PNPLA3 nucleic acid. In certain embodiments, the PNPLA3 inhibitor targets within nucleotides as found in nucleotides 5567-5642, 5644-5731, 5567-5731, 5567-5620 of a PNPLA3 nucleic acid having the nucleobase sequence of SEQ ID NO: 1. In certain embodiments, the PNPLA3 inhibitor is complementary to a site within 5567-5731 of the nucleic acid sequence encoding PNPLA3, e.g., SEQ ID NO: 1. In certain embodiments, the PNPLA3 inhibitor is complementary to a site within 5644-5731 of the nucleic acid sequence encoding PNPLA3, e.g., SEQ ID NO: 1. In certain embodiments, the PNPLA3 inhibitor is complementary to a site within 5567-5642 of the nucleic acid sequence encoding PNPLA3, e.g., SEQ ID NO: 1. In certain embodiments, the PNPLA3 inhibitor is complementary to a site within 5567-5620 of the nucleic acid sequence encoding PNPLA3, e.g., SEQ ID NO: 1. In certain embodiments, these compounds are antisense compounds, oligomeric compounds, or oligonucleotides. In some embodiments, the nucleic acid encoding PNPLA3 is an mRNA.

In certain embodiments, the PNPLA3 inhibitor comprises a modified oligonucleotide 12 to 30 linked nucleosides in length. In certain embodiments, the PNPLA3 inhibitor comprises a modified oligonucleotide 16 to 30 linked nucleosides in length. In certain embodiments, the PNPLA3 inhibitor comprises a modified oligonucleotide 12 to 30 linked nucleosides in length and having a nucleobase sequence comprising at least an 8, 9, 10, 11, 12, 13, 14, 15, or 16 contiguous nucleobase portion any one of SEQ ID NOs: 2, 3, 4, 5, 6, 7, 8, 9 or 10. In certain embodiments, the PNPLA3 inhibitor comprises an antisense oligonucleotide comprising at least 8 contiguous nucleobase of any one of SEQ ID NOs: 2, 3, 4, 5, 6, 7, 8, 9 or 10. In certain embodiments, the modified oligonucleotide is 16 to 30 linked nucleosides in length.

In certain embodiments, the PNPLA3 inhibitor comprises a modified oligonucleotide 12 to 30 linked nucleosides in length and having a nucleobase sequence comprising any one of SEQ ID NOs: 2, 3, 4, 5, 6, 7, 8, 9, or 10. In certain embodiments, the modified oligonucleotide is 16 to 30 linked nucleosides in length. In certain embodiments, the PNPLA3 inhibitor comprises a antisense oligonucleotide comprising a sequence having at least 8, at least 9, at least 10, at least 11, at least 12, at least 13, at least 14, at least 15 or at least 16 contiguous bases of any one of SEQ ID NOs: 2, 3, 4, 5, 6, 7, 8, 9, or 10. In certain embodiments, the PNPLA3 inhibitor comprises a antisense oligonucleotide comprising a sequence having at least 8 contiguous bases of any one of SEQ ID NOs: 2, 3, 4, 5, 6, 7, 8, 9, or 10.

In certain embodiments, the PNPLA3 inhibitor comprises an antisense oligonucleotide comprising any one of SEQ ID NOs: 2, 3, 4, 5, 6, 7, 8, 9 or 10

In certain embodiments, the PNPLA3 inhibitor targeted to PNPLA3 is ION 916333, 975616, 994284, 975605, 994282, 975613, 975617, 975735, 975736, or 975612 as described in U.S. Pat. No. 10,774,333, incorporated by reference herein.

In certain embodiments, any of the foregoing modified oligonucleotides comprises at least one modified internucleoside linkage, at least one modified sugar, and/or at least one modified nucleobase. In certain embodiments, any of the foregoing modified oligonucleotides comprises at least one modified sugar moiety. In some embodiments, the at least one modified sugar moiety is 2′-deoxy, 2′-O-methyl, 2′-O-methoxymethyl, 2′-O-methoxyethyl, 2′-fluoro, 4′-CH (CH₃)—O-2′, 4′—CH₂—O-2′, 4′—(CH₂)₂—O-2′ or combinations thereof. In certain embodiments, at least one modified sugar comprises a 2′-deoxy, 2′-O-methoxyethyl group. In certain embodiments, at least one modified sugar is a bicyclic sugar, such as a 4′-CH (CH₃)—O-2′ group, a 4′-CH₂—O-2′ group, or a 4′-(CH₂)₂—O-2′ group.

In certain embodiments, any of the foregoing modified oligonucleotides comprises one or more modified bases. In some embodiments, the modified base is 5-methylcytosine. In some embodiments, 1, 2, 3, 4, 5, 6 or more cytosine are 5-methylcytosine. In some embodiments, every cytosine in the antisense oligonucleotide is 5-methylcytosine.

In certain embodiments, the modified oligonucleotide comprises at least one modified internucleoside linkage, such as a phosphorothioate internucleoside linkage. In some embodiments, every internucleoside linkage is a phosphorothioate linkage.

In certain embodiments, any of the foregoing modified oligonucleotides comprises: a gap segment consisting of linked deoxynucleosides; a 5′ wing segment consisting of linked nucleosides; and a 3′ wing segment consisting of linked nucleosides; wherein the gap segment is positioned between the 5′ wing segment and the 3′ wing segment and wherein each nucleoside of each wing segment comprises a modified sugar. In certain embodiments, the modified oligonucleotide is 12 to 30 linked nucleosides in length having a nucleobase sequence comprising the sequence recited in any one of SEQ ID NOs: 2, 3, 4, 5, 6, 7, 8, 9, or 10. In certain embodiments, the modified oligonucleotide is 16 to 30 linked nucleosides in length having a nucleobase sequence comprising the sequence recited in any one of SEQ ID NOs: 2, 3, 4, 5, 6, 7, 8, 9, or 10. In certain embodiments, the modified oligonucleotide is 16 linked nucleosides in length having a nucleobase sequence consisting of the sequence recited in any one of SEQ ID NOs: 2, 3, 4, 5, 6, 7, 8, 9, or 10.

In certain embodiments, the PNPLA3 inhibitor is an antisense oligonucleotide comprising:
a gap segment consisting of ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage and wherein each cytosine is a 5-methylcytosine.

In certain embodiments, the PNPLA3 inhibitor is an antisense oligonucleotide comprising of a modified oligonucleotide 12-30 linked nucleobases in length having a nucleobase sequence comprising the sequence recited in any one of SEQ ID NOs: 2, 3, 4, 5, 6, 7, 8, 9, or 10, wherein the modified oligonucleotide comprises a gap segment consisting of ten linked deoxynucleosides;
a 5′ wing segment consisting of three linked nucleosides; and
a 3′ wing segment consisting of three linked nucleosides;
wherein the gap segment is positioned between the 5′ wing segment and the 3′ wing segment,
   wherein each nucleoside of each wing segment comprises a cEt sugar; wherein each internucleoside linkage is a phosphorothioate linkage and wherein each cytosine is a 5-methylcytosine. In certain embodiments, the modified oligonucleotide consists of 16-30 linked nucleosides. In certain embodiments, the modified oligonucleotide consists of 16 linked nucleosides.

In certain embodiments, a compound comprises or consists of a modified oligonucleotide, wherein the modified oligonucleotide is 16 linked nucleosides in length and consists of the sequence of SEQ ID NO: 2, wherein the modified oligonucleotide comprises:
a gap segment consisting of ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage; and wherein each cytosine is a 5-methylcytosine.

In some embodiments, the PNPLA3 inhibitor is an antisense oligonucleotide further comprising a conjugate group. In some embodiments, the conjugate group is at the 5′ end of the antisense oligonucleotide. Thus, in certain embodiments, a compound consists of a modified oligonucleotide and a conjugate group, wherein the modified oligonucleotide is 16 linked nucleosides in length and consists of the sequence of SEQ ID NO: 2, wherein the modified oligonucleotide comprises:
a gap segment consisting of ten linked deoxynucleosides;
a 5′ wing segment consisting of three linked nucleosides; and
a 3′ wing segment consisting of three linked nucleosides;
   wherein the gap segment is positioned between the 5′ wing segment and the 3′ wing segment, wherein each nucleoside of each wing segment comprises a cEt sugar; wherein each internucleoside linkage is a phosphorothioate linkage; wherein each cytosine is a 5-methylcytosine; and wherein the conjugate group is positioned at the 5′ end of the modified oligonucleotide and is

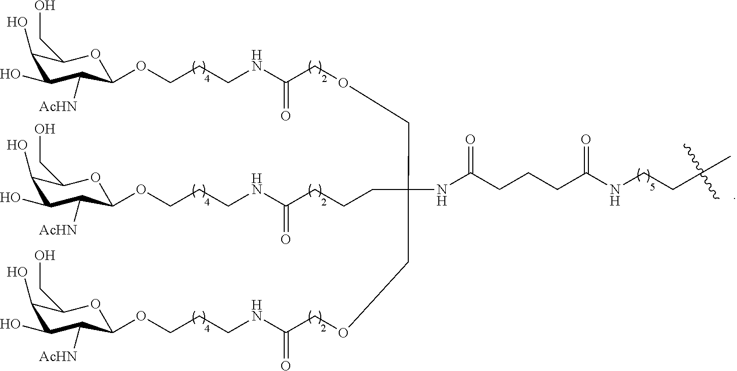

In some embodiments, the inhibitor of PNPLA3 expression is a compound of the following formula (SEQ ID NO: 2):

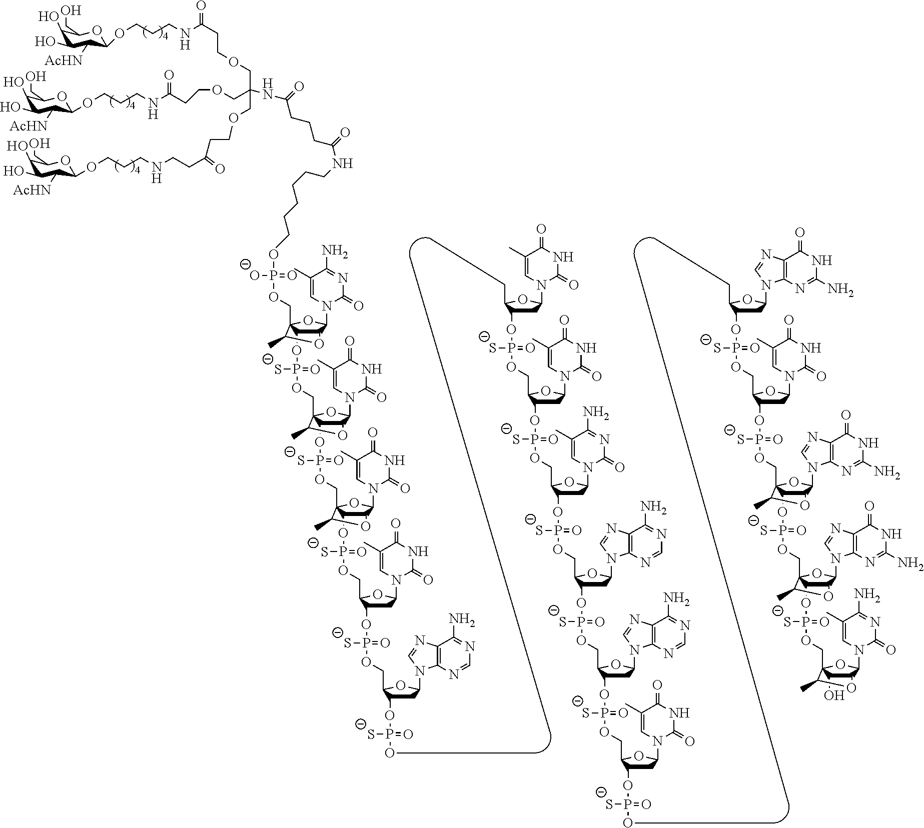

In some embodiments, the inhibitor of PNPLA3 expression is a compound of the following formula (SEQ ID NO: 2):

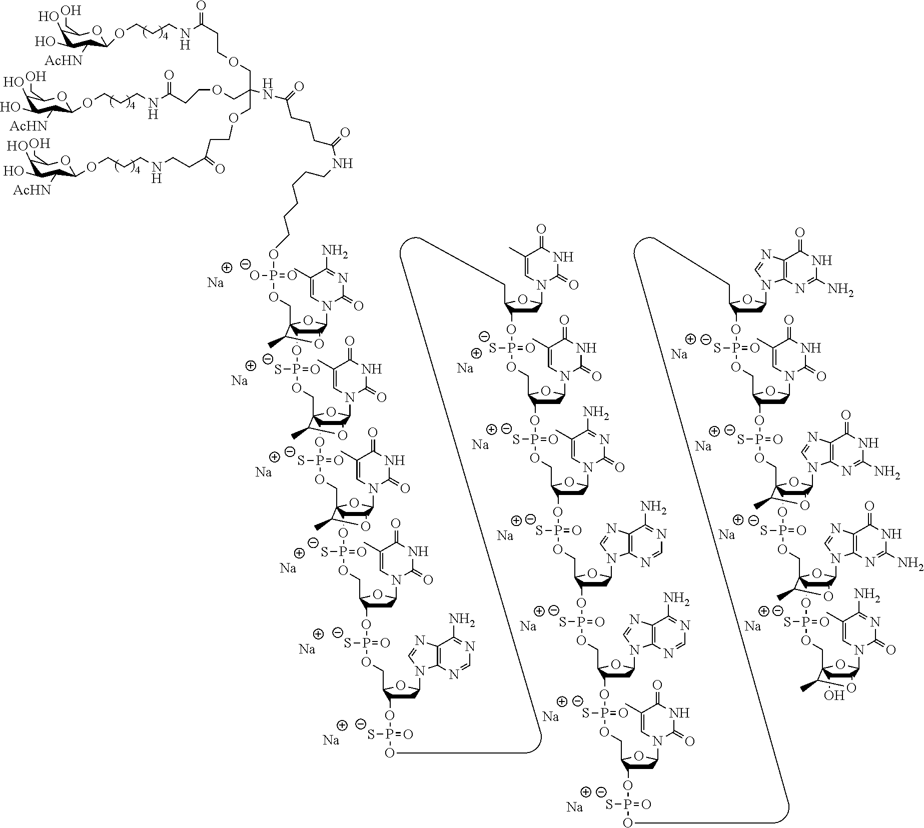

In any of the foregoing embodiments, the PNPLA3 inhibitor is an antisense oligonucleotide, wherein the antisense oligonucleotide can be at least 85%, at least 90%, at least 95%, at least 98%, at least 99%, or 100% complementary to a nucleic acid encoding PNPLA3.

In any of the foregoing embodiments, the PNPLA3 inhibitor is an antisense oligonucleotide, wherein the antisense oligonucleotide can be single-stranded. In certain embodiments, the PNPLA3 inhibitor comprises deoxyribonucleotides. In certain embodiments, the PNPLA3 inhibitor is double-stranded. In certain embodiments, the PNPLA3 inhibitor is double-stranded and comprises ribonucleotides.

In any of the foregoing embodiments, the PNPLA3 inhibitor can be an antisense compound or oligomeric compound.

In any of the foregoing embodiments, the PNPLA3 inhibitor is an antisense oligonucleotide, wherein the antisense oligonucleotide can be 8 to 80, 10 to 30, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked nucleosides in length. In certain embodiments, the PNPLA3 inhibitor is an oligonucleotide.

In certain embodiments, the PNPLA3 inhibitor is an antisense oligonucleotide, wherein the antisense oligonucleotide comprises a modified oligonucleotide described herein and a conjugate group. In certain embodiments, the conjugate group is linked to the modified oligonucleotide at the 5′ end of the modified oligonucleotide. In certain embodiments, the conjugate group is linked to the modified oligonucleotide at the 3′ end of the modified oligonucleotide. In certain embodiments, the conjugate group comprises at least one N-Acetylgalactosamine (GalNAc), at least two N-Acetylgalactosamines (GalNAcs), or at least three N-Acetylgalactosamines (GalNAcs).

In certain embodiments, the PNPLA3 inhibitor provided herein comprise a pharmaceutically acceptable salt of the modified oligonucleotide. In certain embodiments, the salt is a sodium salt. In certain embodiments, the salt is a potassium salt.

In certain embodiments, the PNPLA3 inhibitor as described herein are active by virtue of having at least one of an in vitro IC₅₀ of less than 2 μM, less than 1.5 μM, less than 1 μM, less than 0.9 μM, less than 0.8 μM, less than 0.7 μM, less than 0.6 μM, less than 0.5 μM, less than 0.4 μM, less than 0.3 μM, less than 0.2 μM, less than 0.1 μM, less than 0.05 μM, less than 0.04 μM, less than 0.03 μM, less than 0.02 μM, or less than 0.01 μM.

In certain embodiments, the PNPLA3 inhibitor as described herein are highly tolerable as demonstrated by having at least one of an increase in alanine transaminase (ALT) or aspartate transaminase (AST) value of no more than 4 fold, 3 fold, or 2 fold over control animals, or an increase in liver, spleen, or kidney weight of no more than 30%, 20%, 15%, 12%, 10%, 5%, or 2% compared to control animals. In certain embodiments, the PNPLA3 inhibitor as described herein are highly tolerable as demonstrated by having no increase of ALT or AST over control animals. In certain embodiments, the PNPLA3 inhibitor as described herein are highly tolerable as demonstrated by having no increase in liver, spleen, or kidney weight over control animals.

Certain embodiments provide a composition comprising the PNPLA3 inhibitor of any of the aforementioned embodiments or any pharmaceutically acceptable salt thereof and at least one of a pharmaceutically acceptable carrier or diluent. In certain embodiments, the composition has a viscosity less than about 40 centipoise (cP), less than about 30 centipoise (cP), less than about 20 centipoise (cP), less than about 15 centipoise (cP), or less than about 10 centipoise (cP). In certain embodiments, the composition having any of the aforementioned viscosities comprises a PNPLA3 inhibitor provided herein at a concentration of about 100 mg/mL, about 125 mg/mL, about 150 mg/mL, about 175 mg/mL, about 200 mg/mL, about 225 mg/mL, about 250 mg/mL, about 275 mg/mL, or about 300 mg/mL. In certain embodiments, the composition having any of the aforementioned viscosities and/or PNPLA3 inhibitor concentrations has a temperature of room temperature, or about 20° C., about 21° C., about 22° C., about 23° C., about 24° C., about 25° C., about 26° C., about 27° C., about 28° C., about 29° C., or about 30° C.

### GLP-1 Peptide

Glucagon and glucagon-like peptide-1 (GLP-1) derive from pre-proglucagon, a 158 amino acid precursor polypeptide that is processed in different tissues to form a number of different proglucagon-derived peptides, including glucagon, glucagon-like peptide-1 (GLP-1), glucagon-like peptide-2 (GLP-2) and oxyntomodulin (OXM), that are involved in a wide variety of physiological functions, including glucose homeostasis, insulin secretion, gastric emptying, and intestinal growth, as well as the regulation of food intake. Glucagon is a 29-amino acid peptide that corresponds to amino acids 33 through 61 of proglucagon (53 to 81 of preproglucagon), while GLP-1 is produced as a 37-amino acid peptide that corresponds to amino acids 72 through 108 of proglucagon (92 to 128 of preproglucagon). GLP-1 (7-36) amide or GLP-1 (7-37) acid are biologically active forms of GLP-1, that demonstrate essentially equivalent activity at the GLP-1 receptor. See, e.g., U.S. Pat. No. 9,765,130, incorporated herein by reference.

As used herein a “GLP-1/glucagon agonist peptide” is a chimeric peptide that exhibits activity at the glucagon receptor of at least about 1%, 5%, 10%, 20%, 30%, 40%, 50%, 60%, 70%, 80%, 90%, 95%, or more relative to native glucagon and also exhibits activity at the GLP-1 receptor of about at least about 1%, 5%, 10%, 20%, 30%, 40%, 50%, 60%, 70%, 80%, 90%, 95%, or more relative to native GLP-1, under the conditions of assay 1.

As used herein the term “native glucagon” refers to naturally-occurring glucagon, e.g., human glucagon, comprising the sequence of SEQ ID NO: 11. The term “native GLP-1” refers to naturally-occurring GLP-1, e.g., human GLP-1, and is a generic term that encompasses, e.g., GLP-1 (7-36) amide (SEQ ID NO: 12), GLP-1 (7-37) acid (SEQ ID NO: 13) or a mixture of those two compounds. As used herein, a general reference to “glucagon” or “GLP-1” in the absence of any further designation is intended to mean native human glucagon or native human GLP-1, respectively. Unless otherwise indicated, “glucagon” refers to human glucagon, and “GLP-1” refers to human GLP-1.

Glucagon can be produced by the pancreas and can interact with the glucagon receptor (“GCGR”). Glucagon can act in the liver to raise blood glucose via gluconeogenesis and glycogenolysis. When blood glucose begins to fall, glucagon can signal the liver to break down glycogen and release glucose, causing blood glucose levels to rise toward a normal level. GLP-1 can have different biological activities compared to glucagon. It can be secreted from gut L cells and can bind to the GLP-1 receptor. GLP-1 activities can include stimulation of insulin synthesis and secretion, inhibition of glucagon secretion, and inhibition of food intake.

Provided herein are agonists of glucagon receptor or glucagon-like peptide-1 (GLP-1). In some embodiments, the agonists of glucagon receptor or glucagon-like peptide-1 (GLP-1) comprise a polypeptide. peptides which bind both to a glucagon receptor and to a GLP-1 receptor. In certain embodiments, the peptides provided herein are co-agonists of glucagon and GLP-1 activity. Such peptides are referred to herein as GLP-1/glucagon agonist peptides. GLP-1/glucagon agonist peptides as provided herein possess GLP-1 and glucagon activities with favorable ratios to promote weight loss, prevent weight gain, or to maintain a desirable body weight, and possess optimized solubility, formulatability, and stability. In certain embodiments, GLP-1/glucagon agonist peptides as provided herein are active at the human GLP1 and human glucagon receptors, in certain embodiment relative activity compared to the natural ligand at the GLP-1 receptor is at least about 1-fold, 2-fold 5-fold, 8-fold, 10-fold, 15-fold, 20-fold, or 25-fold higher than at the glucagon receptor.

As used herein, the term “peptide” encompasses a full length peptides and fragments, variants or derivatives thereof, e.g., a GLP-1/glucagon agonist peptide (e.g., 29, 30, or 31 amino acids in length). A “peptide” as disclosed herein, e.g., a GLP-1/glucagon agonist peptide, can be part of a fusion polypeptide comprising additional components such as, e.g., an Fc domain or an albumin domain, to increase half-life. A peptide as described herein can also be derivatized in a number of different ways.

The terms “fragment,” “analog,” “derivative,” or “variant” when referring to a GLP-1/glucagon agonist peptide includes any peptide which retains at least some desirable activity, e.g., binding to glucagon and/or GLP-1 receptors. Fragments of GLP-1/glucagon agonist peptides provided herein include proteolytic fragments, deletion fragments which exhibit desirable properties during expression, purification, and or administration to a subject.

In certain embodiments, GLP-1/glucagon agonist peptides as disclosed have desirable potencies at the glucagon and GLP-1 receptors, and have desirable relative potencies for promoting weight loss. In certain embodiments, GLP-1/glucagon agonist peptides as disclosed exhibit in vitro potencies at the GLP-1 receptor as shown by an EC50 in the CAMP assay 1 (see Example 2) of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM. In certain embodiments, GLP-1/glucagon agonist peptides as disclosed exhibit in vitro potencies at the GLP-1 receptor as shown by EC50 in the CAMP assay in 4.4% human serum albumin (assay 2, see Example 2) of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM. In certain embodiments, GLP-1/glucagon agonist peptides as disclosed exhibit in vitro potencies at the glucagon receptor as shown by an EC50 in the CAMP assay 1 (see Example 2 in U.S. Pat. No. 9,765,130, incorporated by reference herein) of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM. In certain embodiments, GLP-1/glucagon agonist peptides as disclosed exhibit in vitro potencies at the glucagon receptor as shown by an EC50 in the CAMP assay in 4.4% human serum albumin (assay 2, see Example 2 in U.S. Pat. No. 9,765,130, incorporated by reference herein) of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM. In certain embodiments, GLP-1/glucagon agonist peptides as disclosed have relative GLP1-R/GCGR potency ratios, when compared to the native ligands, in the range of about 0.01 to 0.50, e.g., from about 0.02 to 0.30, e.g., about 0.02, 0.03, 0.04, 0.05, 0.06, 0.07, 0.08, 0.09, 0.10, 0.11. 0.12, 0.13, 0.14, 0.15, 0.16, 0.17, 0.18, 0.19, 0.20, 0.21, 0.22, 0.23, 0.24, 0.25, 0.26, 0.27, 0.28, or 0.30. when using assay 2.

In certain embodiments, GLP-1/glucagon agonist peptides as disclosed exhibit in vitro potencies at the glucose-dependent insulinotropic peptide (gastric inhibitory peptide) (GIPR) as shown by an EC50 in the CAMP assay 1 (see Example 2 in U.S. Pat. No. 9,765,130, incorporated by reference herein) of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM. In certain embodiments, GLP-1/glucagon agonist peptides as disclosed exhibit in vitro potencies at the GIPR as shown by EC50 in the CAMP assay in 4.4% human serum albumin (assay 2, see Example 2 in U.S. Pat. No. 9,765,130, incorporated by reference herein) of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM.

In certain embodiments, GLP-1/glucagon agonist peptides provided herein possess one or more criteria of acceptable solubility, ease in formulatability, plasma stability, and improved pharmacokinetic properties. In certain embodiments, GLP-1/glucagon agonist peptides as disclosed are soluble in standard buffers over a broad pH range.

In certain embodiments, GLP-1/glucagon agonist peptides are soluble in common buffer solutions at a concentration up to 0.5 mg/ml, 0.6 mg/ml, 0.7 mg/ml, 0.8 mg/ml, 0.9 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, or more, in buffer systems and a range of ionic strengths, e.g., from 0.25 to 150 mM, including, but not limited to phosphate buffer, Tris buffer, glutamate buffer, acetate buffer, succinate buffer, or histidine buffer. Exemplary buffers include 100 mM glutamate pH 4.5 buffer, 100 mM acetate pH 5 buffer, 100 mM succinate pH 5 buffer, 100 mM phosphate pH 6 buffer, 100 mM histidine pH 6 buffer, 100 mM phosphate pH 6.5 buffer, 100 mM phosphate pH 7.0 buffer, 100 mM histidine pH 7.0 buffer, 100 mM phosphate pH 7.5 buffer, 100 mM Tris pH 7.5 buffer, and 100 mM Tris pH 8.0 buffer. In certain embodiments, GLP-1/glucagon agonist peptides as disclosed are soluble in standard buffers at 0.8 mg/ml over a range of pH, e.g., from pH 4.0 to pH 8.0, e.g., at pH 4.0, 4.5, 5.0, 5.5, 6.0, 6.5, 7.0, 7.5, 8.0, or 8.5. In certain embodiments, GLP-1/glucagon agonist peptides as disclosed are soluble in standard buffers from pH 4.5 to 8.0, 5.0 to 8.0, 5.5 to 8.0, 6.0 to 8.0, 6.5 to 8.0, 7.0 to 8.0, 4.5 to 8.5, 5.5 to 8.5, 5.5 to 8.5, 6.0 to 8.5, 6.5 to 8.5, or 7.0 to 8.5.

In certain embodiments, GLP-1/glucagon agonist peptides as disclosed are formulatable in standard pharmaceutical formulations. Exemplary formulations include, but are not limited to: 0.1M Tris pH 7.5, 150 mM Mannitol, final formulation pH=7.2; 0.05M Tris, 50 mM Arginine/Proline, final formulation pH-8.0; or sodium phosphate buffer (pH8)/1.85% W/V propylene glycol, final formulation pH=7.0. In certain embodiments GLP-1/glucagon agonist peptides as disclosed are soluble is these or other formulations at a concentration up to 0.5 mg/ml, 0.6 mg/ml, 0.7 mg/ml, 0.8 mg/ml, 0.9 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, or more.

In certain embodiments, GLP-1/glucagon agonist peptides as disclosed are acceptably stable against proteases in serum or plasma. Common degradation products of glucagon or GLP-1 include+1 products (acid) and the DPP IV-cleavage products. Products with+1 mass may arise from deamidation at amide groups of glutamine or at the C-terminus Cleavage products arise from the action of the protease DPP IV in plasma. In certain embodiments, GLP-1/glucagon agonist peptides as disclosed are remain stable in plasma at levels up to 30%, 40%, 50%, 60%, 70%, 80%, 90%, or 100% after 24 hours in plasma at 37° C.

Provided herein is a GLP-1/glucagon agonist peptide comprising the amino acid sequence:

HX2QGTFTSDX10SX12X13LX15X16X17X18AX20X21FX23X24WL
X27X28GX30

wherein X2 is G or S, X10 is Y or K, X12 is K, E, R, or S, X13 is K or Y, X15 is D or E. X16 is S or G, X17 is E, R, Q, or K, X18 is R, S, or A, X20 is R, K, or Q, X21 is D or E, X23 is V or I, X24 is A or Q, X27 is E or V, X28 is A or K, and X30 is G or R (SEQ ID NO: 25). In certain embodiments the isolated peptide shown above is provided, where X2 is S, X10 is Y or K, X12 is K, E, R, or S, X13 is K or Y, X15 is D, X16 is S, X17 is E, R, Q, or K, X18 is R, S, or A, X20 is R, X21 is D, X23 is V, X24 is A, X27 is E or V, X28 is A, and X30 is G (SEQ ID NO:26). In certain embodiments the isolated peptide shown above is provided, where X2 is S, X10 is Y or K, X12 is K, E, R, or S, X13 is K or Y, X15 is D, X16 is S, if X17 is E and X18 is R, or if X17 is R and X18 is S, X20 is R, X21 is D, X23 is V, X24 is A, X27 is E or V, X28 is A, and X30 is G (SEQ ID NO: 27 and SEQ ID NO. 28, respectively).

In certain embodiments the isolated peptide shown above is provided, where X2 is S, X10 is Y, X12 is K, X13 is K, X15 is D, X16 is S, if X17 is E and X18 is R, or if X17 is R and X18 is S, X20 is R, X21 is D, X23 is V, X24 is A, X27 is V, X28 is A, and X30 is G (SEQ ID NO: 29 and SEQ ID NO: 30, respectively). In certain embodiments the isolated peptide shown above is provided, where X2 is S, X10 is K, if X12 is K, E, or R and if X12 is K, E, R, or S, X13 is Y, X15 is D, X16 is S, if X17 is E and X18 is R, and if X17 is R and X18 is S, X20 is R, X21 is D, X23 is V, X24 is A, X27 is E, X28 is A, and X30 is G (SEQ ID NO: 31 and SEQ ID NO: 32, respectively). In certain embodiments the isolated peptide shown above is provided, where X2 is S, X10 is K, X12 is E, X13 is Y, X15 is D, X16 is S, if X17 is E and X18 is R, or if X17 is R and X18 is S, X20 is R, X21 is D, X23 is V, X24 is A, X27 is E, X28 is A, and X30 is G (SEQ ID NO: 33 and SEQ ID NO: 34, respectively). In certain embodiments the isolated peptide shown above is provided, where X2 is S, X10 is K, X12 is R, X13 is Y, X15 is D, X16 is S, if X17 is E and X18 is R, or if X17 is R and X18 is S, X20 is R, X21 is D, X23 is V, X24 is A, X27 is E, X28 is A, and X30 is G (SEQ ID NO: 35 and SEQ ID NO: 36, respectively).

GLP-1/glucagon agonist peptides provided herein can include, but are not limited to G730 (SEQ ID NO: 14), G797 (SEQ ID NO: 15), G849 (SEQ ID NO: 16), G933 (SEQ ID NO: 17), G865 (SEQ ID NO: 18), G796 (SEQ ID NO: 19), G812 (SEQ ID NO: 20) and G380 (SEQ ID NO: 21). These GLP-1/glucagon agonist peptides are listed in Table 1:

TABLE 1
GLP-1/Glucagon Peptide Sequence

| | | SEQ ID |
|---|---|---|
| Peptide | Sequence | NO |
| G730 | HSQGT FTSDY SKXD SERAR DFVAW LVAGG-amide X13 = K(gE- | 14 |
| | palm) | |
| G797 | HSQGT FTSDX SEYLD SERAR DFVAW LEAGG-amide X10 = K(gE- | 15 |
| | palm) | |
| G849 | HSQGT FTSDX SRYLD SRSAR DFVAW LEAGG-amide X10 = K(gE- | 16 |
| | palm) | |
| G933 | HSQGT FTSDX SEYLD SERAR DFVAW LEAGG-acid X10 = K(gE- | 17 |
| | palm) | |
| G865 | HSQGT FTSDX SSYLD SRSAR DEVAW LEAGG-amide X10 = K(gE- | 18 |
| | palm) | |
| G796 | HSQGT FTSDX SSYLD SRRAR DFVAW LEAGG-amide X10 = K(gE- | 19 |
| | palm) | |
| G812 | HSQGT FTSDX SKYLE GQAAK EFIAW LEKGR-amide X10 = K(gE- | 20 |
| | palm) | |
| G380 | HGQGT FTSDY SKYLD SXRAQ DFVQW LVAGG-amide X17 = K(gE- | 21 |
| | palm) | |
| G931 | HSQGT FTSDY SKXLD SERAR DFVAW LVAGG-acid X13-K (gE- | 22 |
| | palm) | |
| G934 | HSQGT FTSDX SKYLE GQAAK EFIAW LEKGR-acid X10-K (gE-palm) | 23 |
| G973 | HSQGT FTSDX SSYLD SRSAR DEVAW LEAGG-acid X10-K (gE-palm) | 24 |
| GLP-1 | HAEGT FTSDV SSYLE GQAAK EFIAW LVKGR | 12 |
| (7-36 | | |
| amide) | | |
| GLP-1 | HAEGT FTSDV SSYLE GQAAK EFIAW LVKGRG | 13 |
| (7-37 | | |
| acid) | | |
| Glucagon | HSQGT FTSDY SKYLD SRRAQ DFVQW LMNT | 11 |

i. Methods of Making an Agonist Against Glucagon Receptor and/or GLP-1 Receptors

GLP-1/glucagon agonist peptide. GLP-1/glucagon agonist peptides provided herein can be made by any suitable method, e.g., the methods as described in U.S. Pat. No. 9,765,130, incorporated by reference herein. For example, in certain embodiments the GLP-1/glucagon agonist peptides provided herein are chemically synthesized by methods well known to those of ordinary skill in the art, e.g., by solid phase synthesis as described by Merrifield (1963, J. Am. Chem. Soc. 85:2149-2154). Solid phase peptide synthesis can be accomplished, e.g., by using automated synthesizers, using standard reagents.

Alternatively, GLP-1/glucagon agonist peptides provided herein can be produced recombinantly using a convenient vector/host cell combination as would be well known to the person of ordinary skill in the art. A variety of methods are available for recombinantly producing GLP-1/glucagon agonist peptides. Generally, a polynucleotide sequence encoding the GLP-1/glucagon agonist peptide is inserted into an appropriate expression vehicle, e.g., a vector which contains the necessary elements for the transcription and translation of the inserted coding sequence. The nucleic acid encoding the GLP-1/glucagon agonist peptide is inserted into the vector in proper reading frame. The expression vector is then transfected into a suitable host cell which will express the GLP-1/glucagon agonist peptide. Suitable host cells include without limitation bacteria, yeast, or mammalian cells. A variety of commercially-available host-expression vector systems can be utilized to express the GLP-1/glucagon agonist peptides described herein.

ii. Modifications, Conjugates, Fusions, and Derivations.

In some embodiments, the peptides described herein comprise a modification to an amino acid in the amino acid sequence. In certain embodiments, GLP-1/glucagon agonist peptides provided herein are stabilized via amino acid modifications. In certain embodiments, the carboxyl group of the C-terminal amino acid is amidated. In certain embodiments, the C-terminal amino acid is amidated glycine, e.g., G730, G797, G849, G865, G796, G812, and G380. In certain embodiments, e.g., G933, the C-terminal glycine is the unmodified acid. In certain embodiments, GLP-1/glucagon agonist peptides are provided in which one or more amino acid residues are acylated, i.e., the addition of an acyl moiety. For example, in certain embodiments GLP-1/glucagon agonist peptides provided herein contain one or more lysine residues, in which a palmitoyl moiety is attached to the N (epsilon) group. In certain embodiments a linker is incorporated between lysine and the palmitoyl group. This linker can be a gamma glutamic acid group, or an alternative linker such as, but not limited to, beta alanine and aminohexanoic acid. Different acylation methods may be used such as addition of cholesterol or myristoyl groups. In certain embodiments, the palmitoyl moiety is added at position 13 (e.g., G730). In certain embodiments, the palmitoyl moiety is added at position 10 (e.g., G797, G849, G933, G865, G796, and G812). In certain embodiments, the palmitoyl moiety is added at position 17 (e.g., G380).

The GLP-1/glucagon agonist peptides provided herein, e.g., G730, G797, G849 and G933 can be palmitoylated to extend their half-life by association with serum albumin, thus reducing their propensity for renal clearance, as described in Example 1 of U.S. Pat. No. 9,765,130, incorporated herein by reference.

Alternatively or in addition, a GLP-1/glucagon agonist peptide as disclosed herein can be associated with a heterologous moiety, e.g., to extend half-life. The heterologous moiety can be a protein, a peptide, a protein domain, a linker, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, a cytokine, and a combination of two or more of such moieties.

For example, GLP-1/glucagon agonist peptides can be fused with a heterologous polypeptide. The peptides can be fused to proteins, either through recombinant gene fusion and expression or by chemical conjugation. Proteins that are suitable as partners for fusion include, without limitation, human serum albumin, antibodies and antibody fragments including fusion to the Fc portion of the antibodies. GLP-1 has been fused to these proteins with retention of potency (L. Baggio et al, Diabetes 53 2492-2500 (2004); P. Barrington et al Diabetes, Obesity and Metabolism 13 426-433 (2011); P. Paulik et al American Diabetes Association 2012, Poster 1946). Extended recombinant peptide sequences have also been described to give the peptide high molecular mass (V. Schellenberger et al Nature Biotechnol 27 1186-1190 (2009); PASylation (EP2173890)). In certain embodiments GLP-1/glucagon agonist peptides are incorporated as the N-terminal part of a fusion protein, with the fusion partner, e.g., the albumin or Fc portion, at the C-terminal end. GLP-1/glucagon agonist peptides as described herein can also be fused to peptides or protein domains, such as ‘Albudabs’ that have affinity for human serum albumin (M. S. Dennis et al J Biol Chem 277 35035-35043 (2002); A. Walker et al Protein Eng Design Selection 23 271-278 (2010)). Methods for fusing a GLP-1/glucagon agonist peptides as disclosed herein with a heterologous polypeptide, e.g., albumin or an Fc region, are well known to those of ordinary skill in the art.

Other heterologous moieties can be conjugated to GLP-1/glucagon agonist peptides to further stabilize or increase half-life. For chemical fusion, certain embodiments feature maintenance of a free N-terminus, but alternative points for derivatization can be made. A further alternative method is to derivatize the peptide with a large chemical moiety such as high molecular weight polyethylene glycol (PEG). A “pegylated GLP-1/glucagon agonist peptide” has a PEG chain covalently bound thereto. Derivatization of GLP-1/glucagon agonist peptides, e.g., pegylation, can be done at the lysine that is palmitoylated, or alternatively at a residue such as cysteine, that is substituted or incorporated by extension to allow derivatization. GLP-1/glucagon agonist peptide formats above can be characterized in vitro and/or in vivo for relative potency and the balance between GLP-1 and glucagon receptor activation.

The general term “polyethylene glycol chain” or “PEG chain”, refers to mixtures of condensation polymers of ethylene oxide and water, in a branched or straight chain, represented by the general formula H (OCH₂CH₂),OH, where n is an integer of 3, 4, 5, 6, 7, 8, 9, or more. PEG chains include polymers of ethylene glycol with an average total molecular weight selected from the range of about 500 to about 40,000 Daltons. The average molecular weight of a PEG chain is indicated by a number, e.g., PEG-5,000 refers to polyethylene glycol chain having a total molecular weight average of about 5,000.

PEGylation can be carried out by any of the PEGylation reactions known in the art. See, e.g., Focus on Growth Factors, 3:4-10, 1992 and European patent applications EP 0 154 316 and EP 0 401 384. PEGylation may be carried out using an acylation reaction or an alkylation reaction with a reactive polyethylene glycol molecule (or an analogous reactive water-soluble polymer).

Methods for preparing a PEGylated GLP-1/glucagon agonist peptides generally include the steps of (a) reacting a GLP-1/glucagon agonist peptide or with polyethylene glycol (such as a reactive ester or aldehyde derivative of PEG) under conditions whereby the molecule becomes attached to one or more PEG groups, and (b) obtaining the reaction product(s).

### Administration

The present disclosure provides methods of treating liver disease in a subject by administering to the subject an inhibitor of PNPLA3 expression and an agonist of glucagon receptor and/or GLP-1 receptor. On some embodiments, the inhibitor of PNPLA3 expression and the agonist of glucagon receptor and/or GLP-1 receptor are administered concomitantly. In some embodiments, the inhibitor of PNPLA3 expression and the agonist of glucagon receptor and/or GLP-1 receptor are administered within 1 hour of one another. In some embodiments, the inhibitor of PNPLA3 expression and the agonist of glucagon receptor and/or GLP-1 receptor are administered within 24 hours of one another. In some embodiments, the inhibitor of PNPLA3 expression and the agonist of glucagon receptor and/or GLP-1 receptor are administered within 72 hours of one another. In some embodiments, the inhibitor of PNPLA3 expression and the agonist of glucagon receptor and/or GLP-1 receptor are administered within one week of one another. In some embodiments, the inhibitor of PNPLA3 expression and the agonist of glucagon receptor and/or GLP-1 receptor are administered within two weeks of one another. In some embodiments, the inhibitor of PNPLA3 expression and the agonist of glucagon receptor and/or GLP-1 receptor are administered within one month of one another, within 2 months of one another, or within 3 months or more of one another.

Various modes of administration are known to the skilled artisan and can be used to administer the inhibitor of PNPLA3 expression and the agonist of glucagon receptor and/or GLP-1 receptor. For example, modes of administration can include oral, parenteral, by inhalation or topical. “Parenteral administration” can mean administration through injection or infusion. Parenteral administration can include subcutaneous administration, intravenous administration, intramuscular administration, intraarterial administration, intraperitoneal administration, or intracranial administration, e.g., intrathecal or intracerebroventricular, vaginal or rectal administration. Another example of a form for administration is a solution for injection, in particular for intravenous or intraarterial injection or drip. Inhibitors of PNPLA3 expression and/or GLP-1/glucagon agonist peptides provided herein can be administered as a single dose or as multiple doses. In certain embodiments, and inhibitor of PNPLA3 expression and/or a GLP-1/glucagon agonist peptide is administered by subcutaneous injection.

In some embodiments, the inhibitor of PNPLA3 expression and the agonist of glucagon receptor and/or GLP-1 receptor are administered by the same mode of administration. In some embodiments, the inhibitor of PNPLA3 expression and the agonist of glucagon receptor and/or GLP-1 receptor are administered by different modes of administration. In some embodiments, in the inhibitor of PNPLA3 expression is administered parenterally. In some embodiments, the agonist of glucagon receptor and/or GLP-1 receptor is administered parenterally.

Parenteral formulations can be a single bolus dose, an infusion or a loading bolus dose followed with a maintenance dose. These compositions can be administered at specific fixed or variable intervals, e.g., once a day, or on an “as needed” basis. Dosage regimens also can be adjusted to provide the optimum desired response (e.g., a therapeutic or prophylactic response).

The dosing frequency of the inhibitor of PNPLA3 expression and/or the agonist of glucagon receptor and/or GLP-1 receptor can be determined by the one of ordinary skill in the art without undue experimentation. In some embodiments, the dosing frequency of the inhibitor of PNPLA3 expression is the same as the dosing frequency of the agonist of glucagon receptor and/or GLP-1 receptor. In some embodiments, the dosing frequency of the inhibitor of PNPLA3 expression is different from the dosing frequency of the agonist of glucagon receptor and/or GLP-1 receptor, e.g., is more frequent or less frequent. In some embodiments, the inhibitor of PNPLA3 expression is administered daily, twice daily or three times daily. In some embodiments, the inhibitor of PNPLA3 expression is administered weekly, twice weekly or three times weekly. In some embodiments, the inhibitor of PNPLA3 expression is administered monthly, twice monthly or three times monthly. In some embodiments, the inhibitor of PNPLA3 expression is administered not more than once a week, once every two weeks, once every three weeks, once every 4 weeks, once every five weeks once every six weeks, or once every 7 weeks. In some embodiments, the agonist of glucagon receptor and/or GLP-1 receptor is administered daily, twice daily or three times daily. In some embodiments, the agonist of glucagon receptor and/or GLP-1 receptor is administered weekly, twice weekly or three times weekly. In some embodiments, the agonist of glucagon receptor and/or GLP-1 receptor is administered monthly, twice monthly or three times monthly. In some embodiments, the agonist of glucagon receptor and/or GLP-1 receptor is administered not more than once a week, once every two weeks, once every three weeks, once every 4 weeks, once every five weeks once every six weeks, or once every 7 weeks.

### Indications

The methods, compounds, peptides and compositions described herein can be used to treat liver disease in a subject. The term “subject” is meant any subject, particularly a mammalian subject, in need of treatment with a combination of and inhibitor of PNPLA3 expression and the GLP-1/glucagon agonist peptides provided herein. Mammalian subjects include, but are not limited to, humans, dogs, cats, guinea pigs, rabbits, rats, mice, horses, cattle, bears, cows, apes, monkeys, orangutans, and chimpanzees, and so on. In one embodiment, the subject is a human subject. In some embodiments, the subject is a female subject. In one embodiment, the subject is a male subject.

As used herein, a “subject in need thereof” refers to an individual for whom it is desirable to treat, e.g., a subject having liver disease. In some embodiments, the present disclosure provides a method of treating liver disease, e.g., NASH and or NAFLD. Non-alcoholic fatty liver disease (NAFLD) can include a spectrum of liver disease from steatosis to nonalcoholic steatohepatitis (NASH) and cirrhosis. NAFLD can be defined as fat accumulation in the liver exceeding 5% by weight, in the absence of significant alcohol consumption, steatogenic medication, or hereditary disorders (see, e.g., Kotronen et al, Arterioscler Thromb. Vasc. Biol. 2008, 28:27-38).

Non-alcoholic steatohepatitis (NASH) can be NAFLD with signs of inflammation and hepatic injury. In some embodiments, NASH can be defined histologically by macrovesicular steatosis, hepatocellular ballooning, and lobular inflammatory infiltrates (Sanyal, Hepatol. Res. 2011. 41:670-4). Some studies have estimated that NASH affects 2-3% of the general population. In the presence of other pathologies, such as obesity or diabetes, some studies have reported the estimated prevalence increases to 7% and 62% respectively (see, e.g., Hashimoto et al, J. Gastroenterol. 2011. 46 (1): 63-69).

In some embodiments, the methods provided herein are suitable for treating liver disease, NAFLD, hepatic steatosis, non-alcoholic steatohepatitis (NASH), liver cirrhosis, hepatocellular carcinoma, alcoholic liver disease, alcoholic steatohepatitis (ASH), HCV hepatitis, chronic hepatitis, hereditary hemochromatosis, or primary sclerosing cholangitis. Certain embodiments provided herein are directed to compounds and compositions that reduce liver damage, steatosis, liver fibrosis, liver inflammation, liver scarring or cirrhosis, liver failure, liver enlargement, elevated transaminases, or hepatic fat accumulation in an animal.

In some embodiments, the subject may have a secondary indication, e.g., an obese subject or a subject prone to obesity for whom it is desirable to facilitate weight or body fat loss, weight or body fat maintenance, or to prevent or minimize weight gain over a specified period of time. In some embodiments, the liver disease is non-alcoholic fatty liver disease (NAFLD). In some embodiments, the liver disease is nonalcoholic steatohepatitis. In some embodiments, the liver disease is liver fibrosis.

In some embodiments, the disclosure provides a method of reducing steatosis in the liver of a subject having a liver disease, comprising administering to the subject: i) an inhibitor of patatin like phospholipase domain containing 3 (PNPLA3) expression; and ii) an agonist of glucagon receptor and/or glucagon-like peptide-1 (GLP-1) receptor. In some embodiments, the total liver steatosis is reduced in the subject compared to total liver steatosis when the inhibitor of PNPLA3 expression or the agonist of glucagon receptor GLP-1 receptor is administered alone. In some embodiments, the total liver steatosis is reduced in the subject at least 30% compared to total liver steatosis when the inhibitor of PNPLA3 expression or the agonist of glucagon receptor GLP-1 receptor is administered alone. In some embodiments, the total liver steatosis is reduced in the subject at least 35%, at least 40%, at least 45%, at least 50%, at least 55% or at least 60% compared to total liver steatosis when the inhibitor of PNPLA3 expression or the agonist of glucagon receptor GLP-1 receptor is administered alone.

In some embodiments, the total liver steatosis is reduced in the subject at least 30% compared to total liver steatosis when the inhibitor of PNPLA3 expression or the agonist of glucagon receptor GLP-1 receptor is administered alone. In some embodiments, the total liver steatosis is reduced in the subject at least 35%, at least 40%, at least 45%, at least 50%, at least 55% or at least 60% compared to total liver steatosis when the inhibitor of PNPLA3 expression or the agonist of glucagon receptor GLP-1 receptor is administered alone.

In some embodiments, the disclosure provides a method of reducing inflammation in the liver of a subject having a nonalcoholic fatty liver disease, comprising administering to the subject: i) an inhibitor of patatin like phospholipase domain containing 3 (PNPLA3) expression; and ii) an agonist of glucagon receptor and/or glucagon-like peptide-1 (GLP-1) receptor. In some embodiments, the inflammation in the liver is reduced in the subject at least 50% compared to inflammation in the liver when the inhibitor of PNPLA3 expression or the agonist of glucagon receptor GLP-1 receptor is administered alone. In some embodiments, the inflammation in the liver is reduced in the subject at least 55%, at least 60%, at least 65%, at least 70% or at least 75% compared to inflammation in the liver when the inhibitor of PNPLA3 expression or the agonist of glucagon receptor GLP-1 receptor is administered alone.

In some embodiments, the method of reducing liver collagen in a subject having a liver disease, comprising administering to the subject: i) an inhibitor of patatin like phospholipase domain containing 3 (PNPLA3) expression; and ii) an agonist of glucagon receptor and/or glucagon-like peptide-1 (GLP-1) receptor. In some embodiments, the liver collagen is reduced in the subject at least 25% compared to liver collagen when the inhibitor of PNPLA3 expression or the agonist of glucagon receptor GLP-1 receptor is administered alone. In some embodiments, the liver collagen is reduced in the subject at least 30%, at least 35%, at least 40%, at least 45%, or at least 50% compared to liver collagen when the inhibitor of PNPLA3 expression or the agonist of glucagon receptor GLP-1 receptor is administered alone.

### Pharmaceutical Formulations

In some embodiments, the present disclosure provides a pharmaceutically acceptable composition comprising inhibitor of patatin like phospholipase domain containing 3 (PNPLA3) expression and an agonist of glucagon receptor and/or glucagon-like peptide-1 (GLP-1) receptor and at least one pharmaceutically acceptable excipient.

The terms “composition” or “pharmaceutical composition” refer to compositions containing an inhibitor of PNPLA3 expression and an agonist of glucagon receptor and/or GLP-1 receptor provided herein, along with e.g., pharmaceutically acceptable carriers, excipients, or diluents for administration to a subject in need of treatment, e.g., a human subject with liver disease.

The term “pharmaceutically acceptable” refers to compositions that are, within the scope of sound medical judgment, suitable for contact with the tissues of human beings and animals without excessive toxicity or other complications commensurate with a reasonable benefit/risk ratio. “Pharmaceutical composition” or “pharmaceutical formulation” can include a mixture of substances suitable for administering to an individual. For example, a pharmaceutical composition may comprise one or more compounds or salt thereof and a sterile aqueous solution. In some embodiments, the pharmaceutical formulations comprise a pharmaceutically acceptable carrier or diluent. “Pharmaceutically acceptable carrier or diluent” means any substance suitable for use in administering to an individual. For example, a pharmaceutically acceptable carrier can be a sterile aqueous solution, such as PBS or water-for-injection.

In some embodiments, the pharmaceutical formulations comprise a pharmaceutically acceptable salt. “Pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

Further provided are compositions, e.g., pharmaceutical compositions, that contain an effective amount of an inhibitor of PNPLA3 expression and an agonist of glucagon receptor and/or GLP-1 receptor as provided herein, formulated for the treatment of metabolic diseases, e.g., liver disease. An “effective amount” is that amount of inhibitor of PNPLA3 expression and an agonist of glucagon receptor and/or GLP-1 receptor as provided herein, the administration of which to a subject, either in a single dose or as part of a series, is effective for treatment, e.g., treatment of liver disease. This amount can be a fixed dose for all subjects being treated, or can vary depending upon the weight, health, and physical condition of the subject to be treated, the extent of weight loss or weight maintenance desired, the formulation of peptide, a professional assessment of the medical situation, and other relevant factors.

Compositions of the disclosure can be formulated according to known methods. Suitable preparation methods are described, for example, in Remington's Pharmaceutical Sciences, 19th Edition, A. R. Gennaro, ed., Mack Publishing Co., Easton, Pa. (1995), which is incorporated herein by reference in its entirety. Composition can be in a variety of forms, including, but not limited to an aqueous solution, an emulsion, a gel, a suspension, lyophilized form, or any other form known in the art. In addition, the composition can contain pharmaceutically acceptable additives including, for example, diluents, binders, stabilizers, and preservatives. Once formulated, compositions of the present disclosure can be administered directly to the subject.

Carriers that can be used with compositions of the present disclosure are well known in the art, and include, without limitation, e.g., thyroglobulin, albumins such as human serum albumin, tetanus toxoid, and polyamino acids such as poly L-lysine, poly L-glutamic acid, influenza, hepatitis B virus core protein, and the like. A variety of aqueous carriers can be used, e.g., water, buffered water, 0.8% saline, 0.3% glycine, hyaluronic acid and the like. Compositions can be sterilized by conventional, well known sterilization techniques, or can be sterile filtered. A resulting composition can be packaged for use as is, or lyophilized, the lyophilized preparation being combined with a sterile solution prior to administration. Compositions can contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamineoleate, etc. In some embodiments, the composition is formulated for parenteral administration.

In some embodiments, the disclosure provides for a kit comprising: i) an inhibitor of PNPLA3 expression; and ii) an agonist of glucagon receptor and/or GLP-1 receptor. In some embodiments, the inhibitor of PNPLA3 expression and the agonist of glucagon receptor and/or GLP receptor are in the same dosage form in the kit. In some embodiments, the inhibitor of PNPLA3 expression and the agonist of glucagon receptor and/or GLP-1 receptor are in different dosage form in the kit.

All references cited herein, including patents, patent applications, papers, textbooks and the like, and the references cited therein, to the extent that they are not already, are hereby incorporated herein by reference in their entirety.

### Examples

### Example 1-Combined PNPLA3 silencing inhibition and incretin-based therapy a GLP-1 and Glucagon receptors dual agonist have superior efficacy on improving nonalcoholic fatty liver disease (NAFLD), nonalcoholic steatohepatitis (NASH), and liver fibrosis

### INTRODUCTION

Nonalcoholic fatty liver disease (NAFLD) and its more advanced pathogenic form, nonalcoholic steatohepatitis (NASH), are unmet medical needs that affect a large and growing population (Younossi et 1 Nat Rev Gastroenterol Hepatol, 2018, DOI: 10.1038/nrgastro.2017.109). NAFLD is defined as excess liver fat accumulation (fatty liver) induced by causes other than alcohol intake and includes NAFL and NASH, fibrosis and cirrhosis. Fatty liver progresses to steatohepatitis, NASH, with or without fibrosis in a variable proportion of individuals, ultimately leading to liver cirrhosis, liver failure and hepatocellular carcinoma in susceptible individuals (Friedman et al Nat Med, 2018, DOI: 10.1038/s41591-018-0104-9).

NAFLD and NASH have a strong genetic component. The most common mutation associated with these conditions is the rs738409 variant (148M) of the patatin-like phospholipase domain-containing 3 (PNPLA3) gene (Carlsson et al Aliment Pharmacol Ther, 2020, DOI: 10.1111/apt. 15738). Pnpla3 silencing in the liver improves NAFLD, NASH and associated liver fibrosis in a mouse model genetically engineered to carry the human risk allele variant (148M) in the mouse Pnpla3 gene (Linden et al Mol Metab, 2019, DOI: 10.1016/j.molmet.2019.01.013).

Obesity and type 2 diabetes mellitus (T2DM) are major risk factors for developing NAFLD and NASH and treatments with incretin hormones that decrease body weight and improve glucose homeostasis have been shown to improve NAFLD and NASH. Such incretin hormones include peptide analogs that engage the glucagon-like peptide-1 (GLP-1) receptor, both the GLP-1 and glucagon receptors, or the GLP-1 and gastric inhibitory polypeptide (GIP) receptors (Newsome et al NEJM, 2020, DOI: 10.1056/NEJMoa2028395; Ambry et al Lancet, 2018, DOI: 10.1016/S0140-6736 (18) 30726-8; Boland et al Nat Metab, 2020, DOI: 10.1038/s42255-020-0209-6+Hartman et al Diabetes Care, 2020, DOI: 10.2337/dc19-1892; Kannt et al Diabetes Obes Metab, 2020, DOI: 10.1111/dom.14035).

It was hypothesized that combined treatment with PNPLA3 silencing inhibition and activation of incretin hormone receptors could have an additive beneficial effect on the treatment of NAFLD, NASH, and liver fibrosis. In order to explore this, a genetically engineered mouse model for the human PNPLA3 1148M NASH risk allele was used while fed a NASH-inducing diet. These mice were then treated with; 1) a control antisense oligonucleotide (ASO); 2) a Pnpla3 ASO; 3) Cotadutide, a balanced GLP-1 receptor and glucagon receptor dual agonist peptide; or 4) a combination of both Pnpla3 ASO and Cotadutide.

This study demonstrates for the first time that by combining a PNPLA3 ASO-silencing inhibition treatment with an incretin-mimetic treatment (exemplified by Cotadutide), surprising, superior beneficial treatment effects are achieved on NAFLD, NASH and liver fibrosis. The results achieved by the combination treatment show a synergistic effect compared to the respective treatments on their own.

### Materials and Methods

Murine Pnpla3 cEt 5′-GalNAc3-conjugated cEt ASO and Cotadutide

An optimal potent mouse S-constrained ethyl (cEt)-modified 16-mer ASO targeting the mouse Pnpla3 gene (5′-TATTTTTGGTGTATCC-3′) (SEQ ID NO: 37) was used (Lindén et al Mol Metab, 2019, DOI: 10.1016/j.molmet.2019.01.013). This mouse Pnpla3 ASO was modified by 5′-conjugation with triantennary N-acetylgalactosamine (GalNAc3) to further enhance the liver cell targeting in vivo following subcutaneous administration. The specificity of target knockdown was demonstrated using a chemistry-matched scrambled control GalNAc3-conjugated ASO (5′-GGCCAATACGCCGTCA-3′) (SEQ ID NO: 38). Cotadutide (MEDI0382) has been engineered to balance GLP-1 receptor and glucagon receptor agonism (with a ˜5:1 bias towards GLP-1 receptor affinity) (Henderson et al Diabetes Obes Metab, 2016, DOI: 10.1111/dom.12735).

### Animals

All animal experiments were performed with humane care and were approved by the Gothenburg Ethics Committee for Experimental Animals in Sweden. The holding facility has received full accreditation from the Association for Assessment and Accreditation of Laboratory Animal Care (AAALAC). The human PNPLA3 1148M mutation was introduced into the mouse Pnpla3 gene by replacing the isoleucine codon with a methionine codon in amino acid position 148 of the mouse Pnpla3 gene using homologous recombination as described before (Lindén et al Mol Metab, 2019, DOI: 10.1016/j.molmet.2019.01.013). Heterozygous Pnpla3^{148I/M} mice were intercrossed to generate experimental homozygous Pnpla3^{148M/M} knock-in mice for the pharmacology study. All experimental animals were verified to have the correct genotype using PCR before the study began and verified again using PCR after termination as described before (Linden et al Mol Metab, 2019, DOI: 10.1016/j.molmet.2019.01.013). All animals were housed in transparent makrolon cages with aspen wood chip bedding and nesting material, and the temperature- (21±1° C.) and humidity (50±0%) of the holding facility were controlled. The mice had free access to tap water and food and were on a 12-h day/night cycle.

Male Pnpla3^{148M/M} mice (6-8 weeks of age) were fed a diet high in fat (40 kcal % fat (non-trans fat Primex Shortening), fructose (20 kcal %) and cholesterol (2%) (NASH diet; D16022301, Research Diets, New Brunswick, NJ) for 22 weeks. The mice were then assigned to study groups based on body weight, and fed the same NASH diet and dosed with either 1) control ASO+saline, 2) Pnpla3 ASO+saline, 3) control ASO+Cotadutide, or 4) Pnpla3 ASO+Cotadutide for 14 weeks. The ASOs were dosed at 5 mg/kg/week administered by two subcutaneous injections per week with saline as vehicle. Cotadutide was dosed at 1 nmol/kg administered by daily subcutaneous injections with saline as vehicle. The mice that did not receive Cotadutide treatment were injected with the vehicle (saline) daily so that all animals received the same number of subcutaneous injections. Body weights were recorded during the study. At sacrifice, unfasted mice were euthanized with isoflurane (Forene, Abbot Scandinavia AB, Sweden), blood was collected and plasma isolated, livers were collected, and pieces (same position in the left lateral lobe for all mice) were fixed with 4% formaldehyde in PBS for histology or snap-frozen in liquid N₂ and stored at −80° C.

### Liver Histology

Four μm thick formaldehyde-fixed, paraffin-embedded mouse liver sections (one lover section per mouse) were routinely stained with hematoxylin-eosin (HE). Adjacent sections were immunohistochemically stained for collagen 1A1 (Col1A1, LS-C343921, BioSite, USA). Slides were scanned into Panoramic Scan II (3Dhistech, Hungary), and digital images were analyzed using the image analysis program Visiopharm (version 2020.03.0.7300, Visiopharm, Hørsholm, Denmark) by detecting stained area per total section area for Galectin-3 or Collagen 1A1.

Liver steatosis was determined by evaluation of the HE stained liver sections. Total liver steatosis was determined by measuring the total amount of lipid droplets as a percentage area of the section. Macrovesicular steatosis was determined by measuring the amount of large lipid droplets as a percentage area of the section. Microvesicular steatosis was determined by measuring the amount of small lipid droplets as a percentage area of the section.

Liver macrophages were determined by staining different liver sections with Galectin-3 (Mac2) and determining the percentage of the section stained.

The NAFLD activity score (NAS) was determined according to the method reported by Kleiner et al (Kleiner et al Hepatology, 2005, DOI: 10.1002/hep.20701). The NAS was based on combining the liver steatosis score (steatosis <5%=score 0, 5%-33%=score 1, >33%-66%=score 2, >66%=score 3) and inflammation score (no foci=score 0, <2 foci per 200× field=score 1, 2-4 foci per 200× field=score 2, >4 foci per 200× field=score 3). Hepatocellular ballooning degeneration was not found in any of the mouse livers, and not unexpected since this is rarely observed in preclinical rodent NASH models in contrast to human NASH pathology. All histological assessments were performed by a board-certified veterinary pathologist who was blinded to the treatment.

RNA Preparation and qPCR

RNA was isolated from snap-frozen liver tissue using the RNeasy Mini Kit (Qiagen, Germany). The cDNA templates were generated by reverse transcription with a cDNA kit (ThermoFisher Scientific, Stockholm, Sweden) and used for real-time quantitative PCR with the QuantStudio 7 Flex instrument (Applied Biosystems, Stockholm, Sweden). A commercial complete assay was used to analyze the expression of the mouse Pnpla3 mRNA (Mm00504420_m1, TaqMan, Life Technologies Europe, Stockholm, Sweden). The results were normalized to mouse ribosomal protein large PO (Rplp0, 36B4) with forward primer 5′-GAGGAATCAGATGAGGATATGGGA-3 (SEQ ID NO: 39), reverse primer 5′-AAGCAGGCTGACTTGGTTGC-3′ (SEQ ID NO: 40) and the FAM-TAM-labeled probe 5′-TCGGTCTCTTCGACTAATCCCGCCAA-3′ (SEQ ID NO: 41) (Sigma-Aldrich) as a reference gene.

### Statistical Analysis

Differences between treatment groups were examined using 1-way ANOVA followed by Tukey's post-hoc tests (GraphPad Prism v.8.0.1., GraphPad Software, CA). Differences between treatment groups for the NAFLD activity score (NAS) derived from liver histology were analyzed by ordinal regression analyses followed by correction of family-wise error rate using the Šidák method. A p value less than 0.05 was considered significant. Data are presented as individual values and means±standard errors of the means (SEMs).

### Results and Conclusions

Combined Pnpla3 ASO and Cotadutide treatment have additive effects on the improvement of NAFLD, NASH and liver fibrosis in Pnpla3^{148M/M} mice fed a NASH-inducing diet.

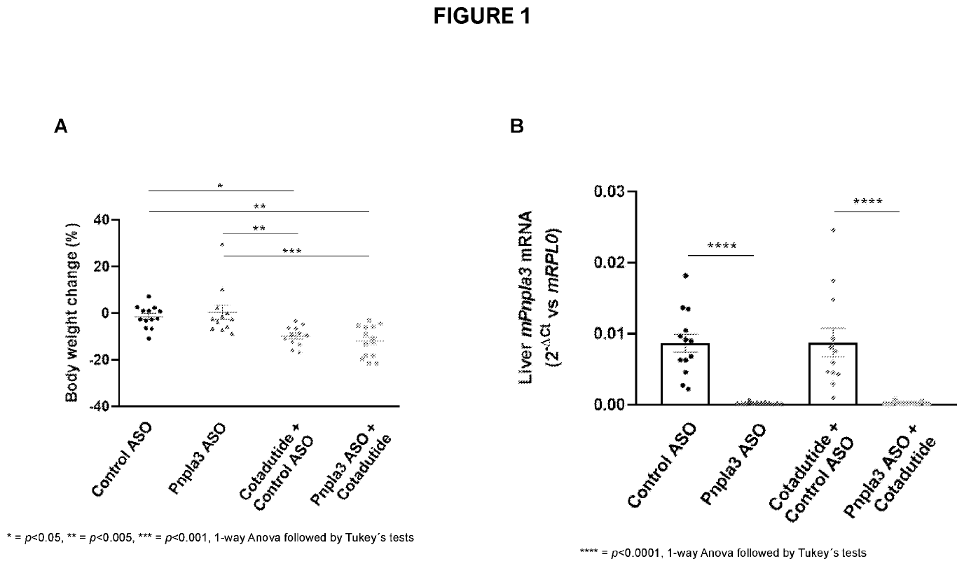

In order to study the combined treatment with Pnpla3 ASO and Cotadutide, homozygous Pnpla3^{148M/M} knock-in mice were fed a NASH-inducing diet for 36 weeks. During the last 14 weeks, they were also treated with either Pnpla3 ASO or Cotadutide alone or in combination and compared with control ASO treated animals (all mice received the same number of subcutaneous injections). Cotadutide treatment, but not Pnpla3 ASO treatment reduced body weight-gain during the treatment period compared to control ASO treated animals (FIG. 1A). Pnpla3 ASO treatment, but not Cotadutide treatment markedly reduced liver Pnpla3 mRNA levels by ≥97% (FIG. 1B).

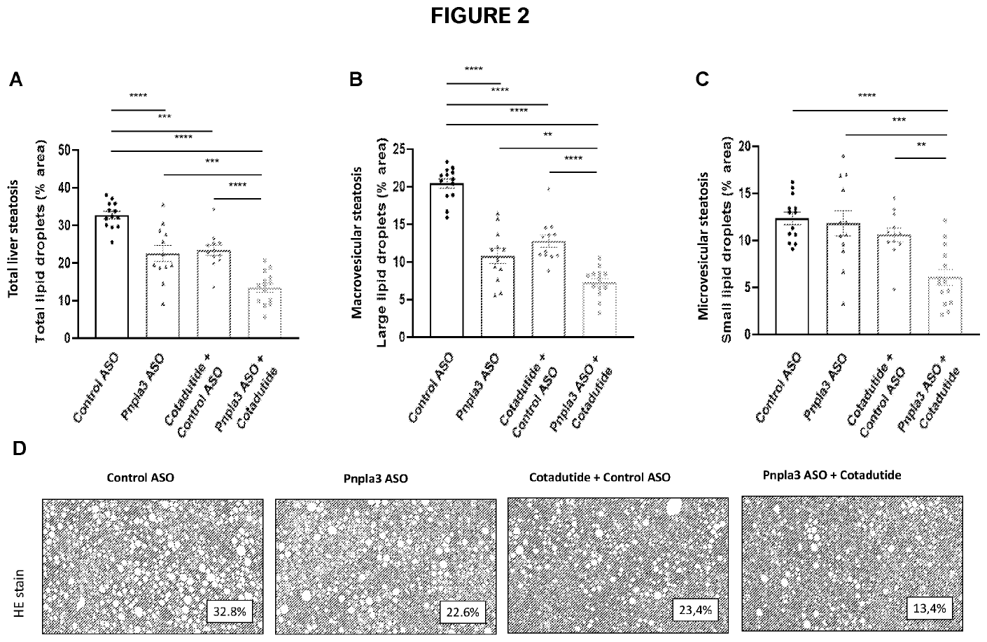

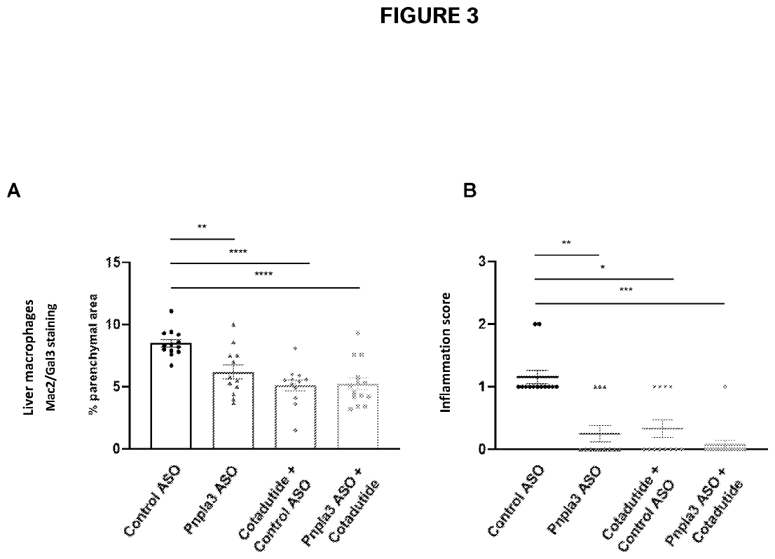

Total liver steatosis (FIG. 2A), macrovesicular steatosis (FIG. 2B) and microvesicular steatosis (FIG. 2C) were determined as described. Stained sections are shown in FIG. 2D, with the percentage of total lipid droplets per area provided for each section. As can be seen in FIGS. 2A-C, combination treatment with Pnpla3 ASO and Cotadutide significantly reduced all types of steatosis compared to either treatment alone. The percentage of liver macrophages (FIG. 3A) and the inflammation score (FIG. 3B) were determined for each treatment as described. As shown in FIGS. 3A and 3B, liver macrophages and inflammation scores were reduced in all treatment arms compared with control.

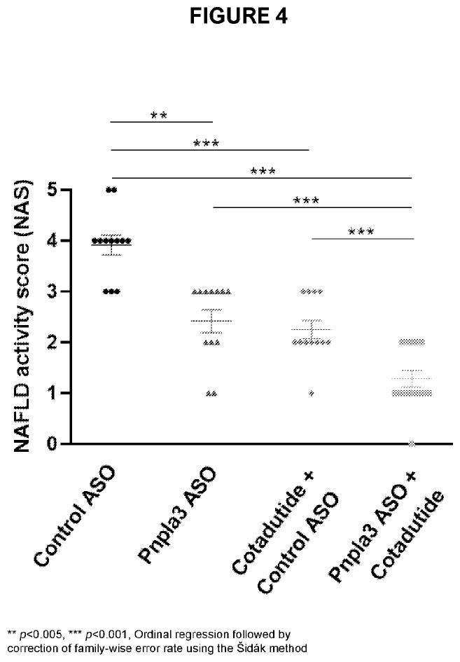

The NAFLD activity score (NAS) was calculated as described above. Pnpla3 ASO treatment reduced the NAS compared to control ASO treated animals (p<0.005) (FIG. 4). Cotadutide treatment also reduced the NAS compared to control ASO treated animals (p<0.001) (FIG. 4). Importantly, combined Pnpla3 ASO and Cotadutide treatment reduced the NAS compared to control ASO treated animals (p<0.001), compared to Pnpla3 ASO treated animals (p<0.001), and compared to Cotadutide treated animals (p<0.001) (FIG. 4). These results show that combined treatment with Pnpla3 knock-down inhibition and a dual GLP-1 and glucagon receptors agonist have an improved beneficial treatment effect on improving NAFLD and NASH.

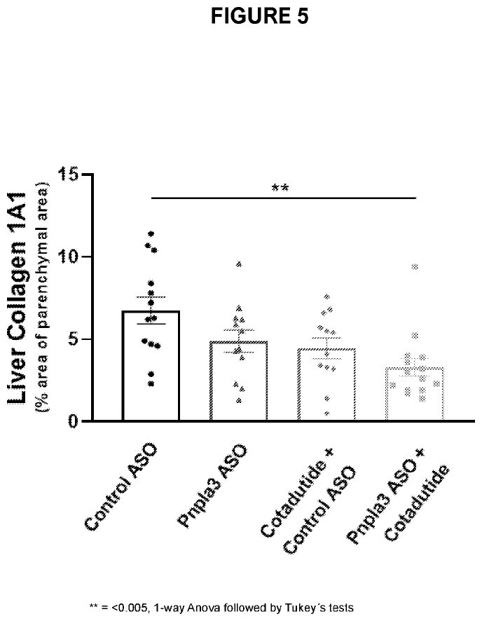

Pnpla3 ASO treatment tended to reduce liver fibrosis measured as liver collagen 1A1 content and also Cotadutide treatment tended to reduce the liver collagen 1A1 content (FIG. 5).

Importantly, combined Pnpla3 ASO and Cotadutide treatment significantly reduced the liver collagen 1A1 content compared to control ASO treated animals (p<0.005) (FIG. 5). Thus, these results show that combined treatment with Pnpla3 knock-down inhibition and a dual GLP-1 and glucagon receptors agonist have a superior beneficial treatment effect on improving liver fibrosis.

What is claimed is:

1. A method of treating a liver disease in a subject, comprising administering to the subject:
i) an inhibitor of patatin like phospholipase domain containing 3 (PNPLA3) expression; and
ii) an agonist of glucagon receptor and/or glucagon-like peptide-1 (GLP-1) receptor.

2. The method of claim 1, wherein the inhibitor of PNPLA3 expression is an antisense oligonucleotide that is complementary to a region of a nucleic acid encoding PNPLA3.

3. The method of claim 2, wherein the antisense oligonucleotide is complementary to a site within nucleotides 5567-5731 of the nucleic acid encoding PNPLA3.

4. The method of claim 2, wherein the antisense oligonucleotide is complementary to a site within nucleotides 5644-5731 of the nucleic acid encoding PNPLA3.

5. The method of claim 2, wherein the antisense oligonucleotide is complementary to a site within nucleotides 5567-5642 of the nucleic acid encoding PNPLA3.

6. The method of claim 2, wherein the antisense oligonucleotide is complementary to a site within nucleotides 5567-5620 of the nucleic acid encoding PNPLA3.

7. The method of any of claims 2 to 6, wherein the nucleic acid encoding PNPLA3 is an mRNA.

8. The method of any of claims 2 to 7, wherein the antisense oligonucleotide is from 12 to 30 nucleosides in length.

9. The method of any of claims 2 to 7, wherein the antisense oligonucleotide is from 16 to 30 nucleosides in length.

10. The method of any of claims 2 to 9, wherein the antisense oligonucleotide comprises one or more modified sugar moieties.

11. The method of claim 10, wherein the one or more modified sugar moieties are 2′-deoxy, 2′-O-methyl, 2′-O-methoxymethyl, 2′-O-methoxyethyl, 2′-fluoro, 4′-CH (CH₃)—O-2′, 4′—CH₂—O-2′, 4′—(CH₂)₂—O-2′ or combinations thereof.

12. The method of any of claims 2 to 11, wherein the antisense oligonucleotide comprises one or more modified bases.

13. The method of claim 12, wherein the one or more modified bases are 5-methylcytosine.

14. The method of claim 13, wherein every cytosine in the antisense oligonucleotide is 5′methylcytosine.

15. The method of any of claims 2 to 14, wherein the antisense oligonucleotide comprises one or more non-natural internucleoside linkages.

16. The method of claim 15, wherein the one or more internucleoside linkages are phosphorothioate linkages.

17. The method of claim 16, wherein every internucleoside linkage is a phosphorothioate linkage.

18. The method of any of claims 2 to 17, wherein the antisense oligonucleotide comprises a sequence having at least 8 contiguous bases of any one of SEQ ID NOs: 2, 3, 4, 5, 6, 7, 8, 9 and 10.

19. The method of any of claims 2 to 17, wherein the antisense oligonucleotide comprises one of SEQ ID NOs: 2, 3, 4, 5, 6, 7, 8, 9 and 10.

20. The method of any of claims 2 to 19, wherein the antisense oligonucleotide comprises:
a) a gap segment consisting of ten linked deoxynucleosides;
b) a 5′ wing segment consisting of three linked nucleosides; and
c) a 3′ wing segment consisting of three linked nucleosides;
wherein the gap segment is positioned between the 5′ wing segment and the 3′ wing segment,
wherein each nucleoside of each wing segment comprises a constrained ethyl sugar,
wherein each internucleoside linkage is a phosphorothioate linkage, and wherein each cytosine is a 5-methylcytosine.

21. The method of any of claims 2 to 20, wherein the inhibitor of the PNPLA3 expression further comprises a conjugate group.

22. The method of claim 21, wherein the conjugate group is at the 5′ end of the antisense oligonucleotide.

23. The method of claim 21 or 22, wherein the conjugate group is:

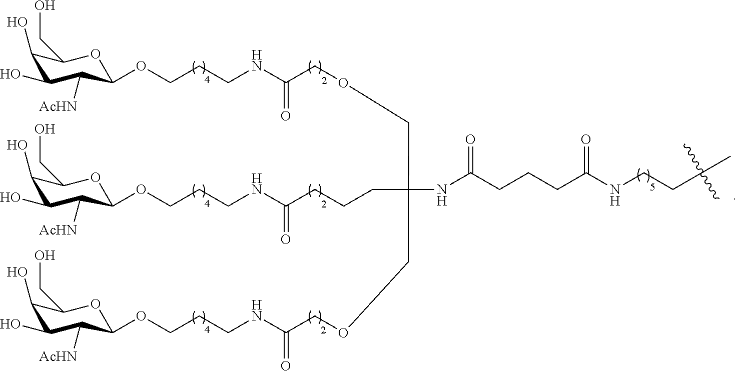

24. The method of any of claims 1 to 23, wherein the inhibitor of PNPLA3 expression is a compound of the following formula (SEQ ID NO: 2): 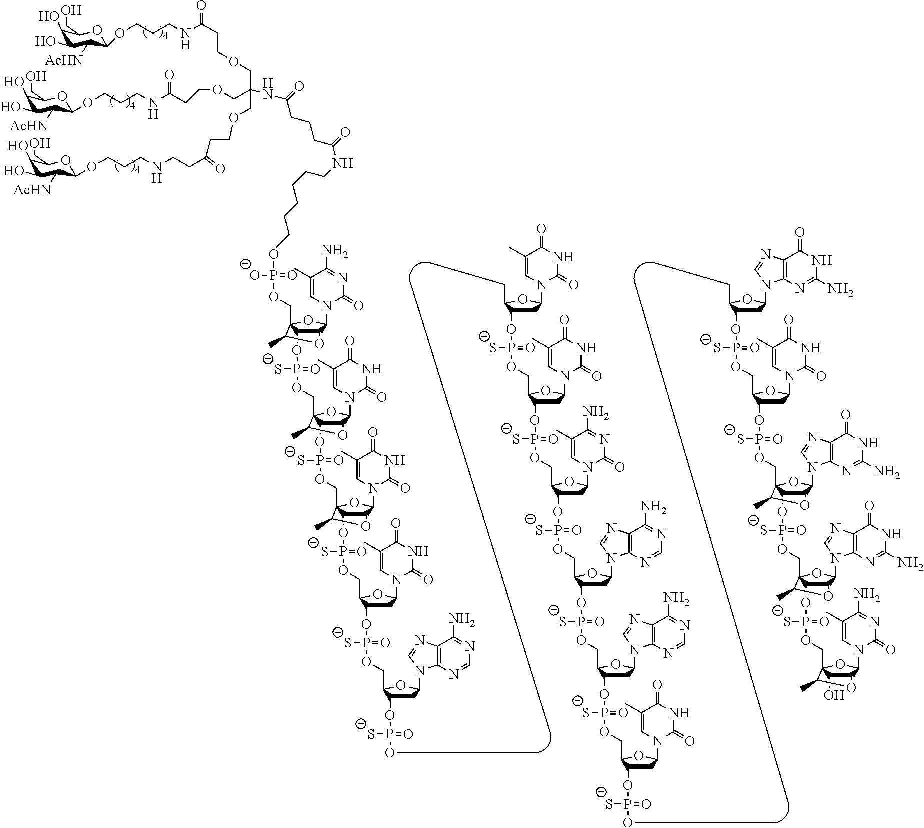
or a pharmaceutically acceptable salt thereof.

25. The method of any of claims 1 to 24, wherein the agonist of glucagon receptor and/or GLP-1 receptor is a peptide.

26. The method of claim 25, wherein the peptide comprises the amino acid sequence:
(SEQ ID NO: 25)
HX2QGTFTSDX10SX12X13LX15X16X17X18AX20X21FX23X24WL
X27X28GX30
wherein,
(1) X2 is S, X10 is Y, X12 is K, X13 is K, X15 is D, X16 is S, X17 is E, X18 is R, X20 is R, X21 is D, X23 is V, X24 is A, X27 is V, X28 is A, and X30 is G (SEQ ID NO: 14;
(2) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 15);
(3) X2 is S, X10 is K, X12 is K, X13 is Y, X15 is E, X16 is G, X17 is Q, X18 is A, X20 is K, X21 is E, X23 is I, X24 is A, X27 is E, X28 is K, and X30 is R (SEQ ID NO: 20);
(4) X2 is S, X10 is K, X12 is S, X13 is Y, X15 is D, X16 is S, X17 is R, X18 is S, X20 is R, X21 is D, X23 is V, X24 is A, X27 is E, X28 is A, and X30 is G (SEQ ID NO: 18);
(5) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 33); or
(6) X2 is S, X10 is K, X12 is S, X13 is Y, X15 is D, X16 is S, X17 is R, X18 is R, X20 is R, X21 is D, X23 is V, X24 is A, X27 is E, X28 is A, and X30 is G (SEQ ID NO:19).

27. The method of claim 25, wherein the peptide comprises the amino acid sequence
(SEQ ID NO: 33)
HSQGTFTSDKSEYLDSERARDFVAWLEAGG.

28. The method of any of claims 25 to 27, wherein the peptide further comprises a modification to an amino acid in the amino acid sequence.

29. The method of claim 28, wherein the modification is the addition of an acyl moiety.

30. The method of claim 29, wherein the modification is a palmitoyl moiety on the N (epsilon) group of a lysine residue.

31. The method of claim 30, wherein the palmitoyl group is linked to the lysine via a linker.

32. The method of claim 31, wherein the linker is gamma glutamic acid.

33. The method of any of claims 1 to 32, wherein the inhibitor of PNPLA3 expression and the agonist of glucagon receptor and/or GLP-1 receptor are administered concomitantly.

34. The method of any of claims 1 to 32, wherein the inhibitor of PNPLA3 expression and the agonist of glucagon receptor and/or GLP-1 receptor are administered within 1 hour of one another.

35. The method of any of claims 1 to 32, wherein the inhibitor of PNPLA3 expression and the agonist of glucagon receptor and/or GLP-1 receptor are administered within 24 hours of one another.

36. The method of any of claims 1 to 32, wherein the inhibitor of PNPLA3 expression and the agonist of glucagon receptor and/or GLP-1 receptor are administered within 72 hours of one another.

37. The method of any of claims 1 to 32, wherein the inhibitor of PNPLA3 expression and the agonist of glucagon receptor and/or GLP-1 receptor are administered within one week of one another.

38. The method of any of claims 1 to 32, wherein the inhibitor of PNPLA3 expression and the agonist of glucagon receptor and/or GLP-1 receptor are administered within two weeks of one another.

39. The method of any of claims 1 to 38, wherein the inhibitor of PNPLA3 expression is administered parenterally.

40. The method of any of claims 1 to 39, wherein the inhibitor of PNPLA3 expression is administered daily, twice daily or three times daily.

41. The method of any of claims 1 to 39, wherein the inhibitor of PNPLA3 expression is administered weekly, twice weekly or three times weekly.

42. The method of any of claims 1 to 39, wherein the inhibitor of PNPLA3 expression is administered monthly, twice monthly or three times monthly.

43. The method of any of claims 1 to 42, wherein the agonist of glucagon receptor and/or GLP-1 receptor is administered parenterally.

44. The method of any of claims 1 to 42, wherein the agonist of glucagon receptor and/or GLP-1 receptor is administered daily, twice daily or three times daily.

45. The method of any of claims 1 to 42, wherein the agonist of glucagon receptor and/or GLP-1 receptor is administered weekly, twice weekly or three times weekly.

46. The method of any of claims 1 to 42, wherein the agonist of glucagon receptor and/or GLP-1 receptor is administered monthly, twice monthly or three times monthly.

47. The method of any of claims 1 to 46, wherein the subject is obese and/or has type 2 diabetes mellitus.

48. The method of any of claims 1 to 47, wherein the liver disease is non-alcoholic fatty liver disease (NAFLD).

49. The method of any of claims 1 to 47, wherein the liver disease is nonalcoholic steatohepatitis.

50. The method of any of claims 1 to 47, wherein the liver disease is liver fibrosis.

51. A method of reducing steatosis in the liver of a subject having a liver disease, comprising administering to the subject:
i) an inhibitor of patatin like phospholipase domain containing 3 (PNPLA3) expression; and
ii) an agonist of glucagon receptor and/or glucagon-like peptide-1 (GLP-1) receptor.

52. The method of claim 51, wherein the inhibitor of PNPLA3 expression is an antisense oligonucleotide that is complementary to a region of a nucleic acid encoding PNPLA3.

53. The method of claim 52, wherein the antisense oligonucleotide is from 12 to 30 nucleosides in length.

54. The method of claim 52, wherein the antisense oligonucleotide is from 16 to 30 nucleosides in length.

55. The method of any of claims 52 to 54, wherein the antisense oligonucleotide comprises a sequence having at least 8 contiguous bases of any one of SEQ ID NOs: 2, 3, 4, 5, 6, 7, 8, 9 and 10.

56. The method of any of claims 52 to 54, wherein the antisense oligonucleotide comprises one of SEQ ID NOs: 2, 3, 4, 5, 6, 7, 8, 9 and 10.

57. The method of any of claims 52 to 56, wherein the antisense oligonucleotide comprises:
a) a gap segment consisting of ten linked deoxynucleosides;
b) a 5′ wing segment consisting of three linked nucleosides; and
c) a 3′ wing segment consisting of three linked nucleosides;
wherein the gap segment is positioned between the 5′ wing segment and the 3′ wing segment, wherein each nucleoside of each wing segment comprises a constrained ethyl sugar;
wherein each internucleoside linkage is a phosphorothioate linkage and wherein each cytosine is a 5-methylcytosine.

58. The method of any of claims 51 to 57, wherein the agonist of glucagon receptor and/or GLP-1 receptor is a peptide.

59. The method of claim 58, wherein the peptide comprises the amino acid sequence:
(SEQ ID NO: 25)
HX2QGTFTSDX10SX12X13LX15X16X17X18AX20X21FX23X24WL
X27X28GX30
wherein,
(1) X2 is S, X10 is Y, X12 is K, X13 is K, X15 is D, X16 is S, X17 is E, X18 is R, X20 is R, X21 is D, X23 is V, X24 is A, X27 is V, X28 is A, and X30 is G (SEQ ID NO: 14);
(2) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 15);
(3) X2 is S, X10 is K, X12 is K, X13 is Y, X15 is E, X16 is G, X17 is Q, X18 is A, X20 is K, X21 is E, X23 is I, X24 is A, X27 is E, X28 is K, and X30 is R (SEQ ID NO: 20);
(4) X2 is S, X10 is K, X12 is S, X13 is Y, X15 is D, X16 is S, X17 is R, X18 is S, X20 is R, X21 is D, X23 is V, X24 is A, X27 is E, X28 is A, and X30 is G (SEQ ID NO: 18);
(5) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 33); or
(6) X2 is S, X10 is K, X12 is S, X13 is Y, X15 is D, X16 is S, X17 is R, X18 is R, X20 is R, X21 is D, X23 is V, X24 is A, X27 is E, X28 is A, and X30 is G (SEQ ID NO: 19).

60. The method of any one of claims 51 to 59, wherein total liver steatosis is reduced in the subject compared to total liver steatosis when the inhibitor of PNPLA3 expression or the agonist of glucagon receptor GLP-1 receptor is administered alone.

61. The method of any one of claims 51 to 59, wherein total liver steatosis is reduced in the subject at least 30% compared to total liver steatosis when the inhibitor of PNPLA3 expression or the agonist of glucagon receptor GLP-1 receptor is administered alone.

62. The method of any one of claims 51 to 59, wherein total liver steatosis is reduced in the subject at least 30% compared to total liver steatosis when the inhibitor of PNPLA3 expression or the agonist of glucagon receptor GLP-1 receptor is administered alone.

63. The method of any of claims 51 to 62, wherein the liver disease is non-alcoholic fatty liver disease (NAFLD).

64. The method of any of claims 51 to 62, wherein the liver disease is nonalcoholic steatohepatitis.

65. The method of any of claims 51 to 62, wherein the liver disease is liver fibrosis.

66. A method of reducing inflammation in the liver of a subject having a nonalcoholic fatty liver disease, comprising administering to the subject:
i) an inhibitor of patatin like phospholipase domain containing 3 (PNPLA3) expression; and
ii) an agonist of glucagon receptor and/or glucagon-like peptide-1 (GLP-1) receptor.

67. The method of claim 66, wherein the inhibitor of PNPLA3 expression is an antisense oligonucleotide that is complementary to a region of a nucleic acid encoding PNPLA3.

68. The method of claim 67, wherein the antisense oligonucleotide is from 12 to 30 nucleosides in length.

69. The method of claim 67, wherein the antisense oligonucleotide is from 16 to 30 nucleosides in length.

70. The method of any of claims 67 to 69, wherein the antisense oligonucleotide comprises a sequence having at least 8 contiguous bases of any one of SEQ ID NOs: 2, 3, 4, 5, 6, 7, 8, 9 and 10.

71. The method of any of claims 67 to 69, wherein the antisense oligonucleotide comprises one of SEQ ID NOs: 2, 3, 4, 5, 6, 7, 8, 9 and 10.

72. The method of any of claims 67 to 71, wherein the antisense oligonucleotide comprises:
a) a gap segment consisting of ten linked deoxynucleosides;
b) a 5′ wing segment consisting of three linked nucleosides; and
c) a 3′ wing segment consisting of three linked nucleosides;
wherein the gap segment is positioned between the 5′ wing segment and the 3′ wing segment, wherein each nucleoside of each wing segment comprises a constrained ethyl sugar;
wherein each internucleoside linkage is a phosphorothioate linkage and wherein each cytosine is a 5-methylcytosine.

73. The method of any of claims 66 to 72, wherein the agonist of glucagon receptor and/or GLP-1 receptor is a peptide.

74. The method of claim 73, wherein the peptide comprises the amino acid sequence:
(SEQ ID NO: 25)
HX2QGTFTSDX10SX12X13LX15X16X17X18AX20X21FX23X24WL
X27X28GX30
wherein,
(1) X2 is S, X10 is Y, X12 is K, X13 is K, X15 is D, X16 is S, X17 is E, X18 is R, X20 is R, X21 is D, X23 is V, X24 is A, X27 is V, X28 is A, and X30 is G (SEQ ID NO: 14);
(2) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 15);
(3) X2 is S, X10 is K, X12 is K, X13 is Y, X15 is E, X16 is G, X17 is Q, X18 is A, X20 is K, X21 is E, X23 is I, X24 is A, X27 is E, X28 is K, and X30 is R (SEQ ID NO: 20);
(4) X2 is S, X10 is K, X12 is S, X13 is Y, X15 is D, X16 is S, X17 is R, X18 is S, X20 is R, X21 is D, X23 is V, X24 is A, X27 is E, X28 is A, and X30 is G (SEQ ID NO: 18);
(5) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 33); or
(6) X2 is S, X10 is K, X12 is S, X13 is Y, X15 is D, X16 is S, X17 is R, X18 is R, X20 is R, X21 is D, X23 is V, X24 is A, X27 is E, X28 is A, and X30 is G (SEQ ID NO:19).

75. The method of any one of claims 50 to 58, wherein inflammation in the liver is reduced in the subject at least 50% compared to inflammation in the liver when the inhibitor of PNPLA3 expression or the agonist of glucagon receptor GLP-1 receptor is administered alone.

76. A method of reducing liver collagen in a subject having a liver disease, comprising administering to the subject:
i) an inhibitor of patatin like phospholipase domain containing 3 (PNPLA3) expression; and
ii) an agonist of glucagon receptor and/or glucagon-like peptide-1 (GLP-1) receptor.

77. The method of claim 76, wherein the inhibitor of PNPLA3 expression is an antisense oligonucleotide that is complementary to a region of a nucleic acid encoding PNPLA3.

78. The method of claim 77, wherein the antisense oligonucleotide is from 12 to 30 nucleosides in length.

79. The method of claim 77, wherein the antisense oligonucleotide is from 16 to 30 nucleosides in length.

80. The method of any of claims 77 to 79, wherein the antisense oligonucleotide comprises a sequence having at least 8 contiguous bases of any one of SEQ ID NOs: 2, 3, 4, 5, 6, 7, 8, 9 and 10.

81. The method of any of claims 77 to 79, wherein the antisense oligonucleotide comprises one of SEQ ID NOs: 2, 3, 4, 5, 6, 7, 8, 9 and 10.

82. The method of any of claims 77 to 81, wherein the antisense oligonucleotide comprises:
a) a gap segment consisting of ten linked deoxynucleosides;
b) a 5′ wing segment consisting of three linked nucleosides; and
c) a 3′ wing segment consisting of three linked nucleosides;
wherein the gap segment is positioned between the 5′ wing segment and the 3′ wing segment, wherein each nucleoside of each wing segment comprises a constrained ethyl sugar;
wherein each internucleoside linkage is a phosphorothioate linkage and wherein each cytosine is a 5-methylcytosine.

83. The method of any of claims 76 to 82, wherein the agonist of glucagon receptor and/or GLP-1 receptor is a peptide.

84. The method of claim 83, wherein the peptide comprises the amino acid sequence:
(SEQ ID NO: 25)
HX2QGTFTSDX10SX12X13LX15X16X17X18AX20X21FX23X24WL
X27X28GX30
wherein,
(1) X2 is S, X10 is Y, X12 is K, X13 is K, X15 is D, X16 is S, X17 is E, X18 is R, X20 is R, X21 is D, X23 is V, X24 is A, X27 is V, X28 is A, and X30 is G (SEQ ID NO: 14);
(2) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 15);
(3) X2 is S, X10 is K, X12 is K, X13 is Y, X15 is E, X16 is G, X17 is Q, X18 is A, X20 is K, X21 is E, X23 is I, X24 is A, X27 is E, X28 is K, and X30 is R (SEQ ID NO: 20);
(4) X2 is S, X10 is K, X12 is S, X13 is Y, X15 is D, X16 is S, X17 is R, X18 is S, X20 is R, X21 is D, X23 is V, X24 is A, X27 is E, X28 is A, and X30 is G (SEQ ID NO: 18);
(5) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 33); or
(6) X2 is S, X10 is K, X12 is S, X13 is Y, X15 is D, X16 is S, X17 is R, X18 is R, X20 is R, X21 is D, X23 is V, X24 is A, X27 is E, X28 is A, and X30 is G (SEQ ID NO:19).

85. The method of any one of claims 50 to 84, wherein liver collagen is reduced in the subject at least 25% compared to liver collagen when the inhibitor of PNPLA3 expression or the agonist of glucagon receptor GLP-1 receptor is administered alone.

86. The method of any of claims 50 to 85, wherein the subject is obese and/or has type 2 diabetes mellitus.

87. The method of any of claims 50 to 85, wherein the liver disease is nonalcoholic steatohepatitis.

88. The method of any of claims 50 to 85, wherein the liver disease is liver fibrosis.

89. A pharmaceutically acceptable composition comprising:
i) an inhibitor of patatin like phospholipase domain containing 3 (PNPLA3) expression;
ii) an agonist of glucagon receptor and/or glucagon-like peptide-1 (GLP-1) receptor;
iii) at least one pharmaceutically acceptable excipient.

90. The composition of claim 89, wherein the inhibitor of PNPLA3 expression is an antisense oligonucleotide that is complementary to a region of a nucleic acid encoding PNPLA3.

91. The composition of claim 90, wherein the antisense oligonucleotide comprises a sequence having at least 8 contiguous bases of any one of SEQ ID NOs: 2, 3, 4, 5, 6, 7, 8, 9 and 10.

92. The composition of claim 90, wherein the antisense oligonucleotide comprises one of SEQ ID NOs: 2, 3, 4, 5, 6, 7, 8, 9 and 10.

93. The composition of any of claims 90 to 92, wherein the antisense oligonucleotide comprises:
a) a gap segment consisting of ten linked deoxynucleosides;
b) a 5′ wing segment consisting of three linked nucleosides; and
c) a 3′ wing segment consisting of three linked nucleosides;
wherein the gap segment is positioned between the 5′ wing segment and the 3′ wing segment, wherein each nucleoside of each wing segment comprises a constrained ethyl sugar;
wherein each internucleoside linkage is a phosphorothioate linkage and wherein each cytosine is a 5-methylcytosine.

94. The composition of any of claims 89 to 93, wherein the agonist of glucagon receptor and/or GLP-1 receptor is a peptide.

95. The composition of claim 94, wherein the peptide comprises the amino acid sequence:
(SEQ ID NO: 25)
HX2QGTFTSDX10SX12X13LX15X16X17X18AX20X21FX23X24WL
X27X28GX30
wherein,
(1) X2 is S, X10 is Y, X12 is K, X13 is K, X15 is D, X16 is S, X17 is E, X18 is R, X20 is R, X21 is D, X23 is V, X24 is A, X27 is V, X28 is A, and X30 is G (SEQ ID NO: 14);
(2) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 15);
(3) X2 is S, X10 is K, X12 is K, X13 is Y, X15 is E, X16 is G, X17 is Q, X18 is A, X20 is K, X21 is E, X23 is I, X24 is A, X27 is E, X28 is K, and X30 is R (SEQ ID NO: 20);
(4) X2 is S, X10 is K, X12 is S, X13 is Y, X15 is D, X16 is S, X17 is R, X18 is S, X20 is R, X21 is D, X23 is V, X24 is A, X27 is E, X28 is A, and X30 is G (SEQ ID NO: 18); (5) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 33); or
(6) X2 is S, X10 is K, X12 is S, X13 is Y, X15 is D, X16 is S, X17 is R, X18 is R, X20 is R, X21 is D, X23 is V, X24 is A, X27 is E, X28 is A, and X30 is G (SEQ ID NO:19).

96. The composition of any of claims 89 to 95, wherein the composition is formulated for parenteral administration.

97. A kit comprising:
i) an inhibitor of patatin like phospholipase domain containing 3 (PNPLA3) expression; and
ii) an agonist of glucagon receptor and/or glucagon-like peptide-1 (GLP-1) receptor.

98. The kit of claim 97, wherein the inhibitor of PNPLA3 expression is an antisense oligonucleotide that is complementary to a region of a nucleic acid encoding PNPLA3.

99. The kit of claim 98, wherein the antisense oligonucleotide comprises a sequence having at least 8 contiguous bases of any one of SEQ ID NOs: 2, 3, 4, 5, 6, 7, 8, 9 and 10.

100. The kit of claim 98, wherein the antisense oligonucleotide comprises one of SEQ ID NOs: 2, 3, 4, 5, 6, 7, 8, 9 and 10.

101. The kit of any of claims 98 to 100, wherein the antisense oligonucleotide comprises:
a) a gap segment consisting of ten linked deoxynucleosides;
b) a 5′ wing segment consisting of three linked nucleosides; and
c) a 3′ wing segment consisting of three linked nucleosides;
wherein the gap segment is positioned between the 5′ wing segment and the 3′ wing segment, wherein each nucleoside of each wing segment comprises a constrained ethyl sugar; wherein each internucleoside linkage is a phosphorothioate linkage and wherein each cytosine is a 5-methylcytosine.

102. The kit of any of claims 97 to 101, wherein the agonist of glucagon receptor and/or GLP-1 receptor is a peptide.

103. The kit of claim 102, wherein the peptide comprises the amino acid sequence:
(SEQ ID NO: 25)
HX2QGTFTSDX10SX12X13LX15X16X17X18AX20X21FX23X24WL
X27X28GX30
wherein,
(1) X2 is S, X10 is Y, X12 is K, X13 is K, X15 is D, X16 is S, X17 is E, X18 is R, X20 is R, X21 is D, X23 is V, X24 is A, X27 is V, X28 is A, and X30 is G (SEQ ID NO: 14);
(2) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 15);
(3) X2 is S, X10 is K, X12 is K, X13 is Y, X15 is E, X16 is G, X17 is Q, X18 is A, X20 is K, X21 is E, X23 is I, X24 is A, X27 is E, X28 is K, and X30 is R (SEQ ID NO: 20);
(4) X2 is S, X10 is K, X12 is S, X13 is Y, X15 is D, X16 is S, X17 is R, X18 is S, X20 is R, X21 is D, X23 is V, X24 is A, X27 is E, X28 is A, and X30 is G (SEQ ID NO: 18);
(5) X2 is S, X10 is K, X12 is E, X13 is Y, X15 is D, X16 is S, X17 is E, X18 is R, X20 is R, X21 is D, X23 is V, X24 is A, X27 is E, X28 is A, and X30 is G (SEQ ID NO: 33); or
(6) X2 is S, X10 is K, X12 is S, X13 is Y, X15 is D, X16 is S, X17 is R, X18 is R, X20 is R, X21 is D, X23 is V, X24 is A, X27 is E, X28 is A, and X30 is G (SEQ ID NO:19).


SEQUENCE LISTING (41 sequences)

SEQ ID NO: 1 - DNA, 33500 bp, Homo sapiens
gaacactgagttcaagataagaaatgtttataactctgtgctggcagccaagagctggatcccagttgcagatggggctatgcgtagcagtgagagtgggagacacagagacagggactgagagagagagagagagagatgggatcagggataagggcagtactgccaggcactgggctgggtgctttctgttcatcacatttaacttcacaatgcccctcacacaactgttttctcccccatggtacagatataaaaagtgaagctctgagagatgaaatgatgcttgcgagtcttgtgcactggctgtctgatgccaaagcttgcagtctcaaccatgatatcatgtgcctgtcccactggaggctggatggggggctgagagagctggaccatgaaacccttgcccagctgggtcctctctttgtctctggaaagtggggagggcccccagctgctgtctacatgcagtgacgaagagggggtgagtcctcagccctgctcctgtcctgcccacatctgggtcacactggggatgacagatgtccagtgtggctggccaagaatgaatgccggtggacaggaagagaatatgaatgcctgtggctggtggcagggtgactcatctcgggccttcggtcacagcctcaaattcctgtgccagagcctggccctcctgtcattagtcctaagactggtcatgcaatgcaggagggatggatgggtacaggaggctagaggttctgaccccagctgtcccctccagaagcctcagttccgccgcagccctgagcgtgaggtgggggtggagggcagttagggcaggactgtaggtaatgcccctctccagcaaagccaacacgtcaggaatagatgggaccccctgtctgcttctcagctctcctgaccttccccgggacttaggacctgaccctgccttgctctgggcctccatctacaaagtgtctatgtgatgaggcagtcagcccaggtattcactaaggcctgaagctcccatgccctgggattcctgcccagagaaaggaggcagccctgctcccagctcccctacccctccaggcaaggcaaaagcaaggactgcagcagaatggggaagctcagttgaacatgaaccacccaaccaatccaggtgcttctcaggtgacatttggaatcctgacagcaatgtcacctgttttacaacaccaccagtcattctgcagttgaggacactgagccacaaagagggcagaggcctgaccccaaagctcacatactctgcattcctccaggtctcacggagaagggggagggcttcctggtccactccagctgggggcgctatggcccggcagaccagggtcctccagtgaggagctctggaccaaggtctaacttggctccatgtactgctgtgggcttgctccacccagtgccaggggtaggtcaaggtcaaggggcctcttaggcatctcctaagggcatgctactatccaaatgtttgtgtccccactaaaggtcatctgttgaaatcctacaccttaaggtgatgatctttggaggcgggggcctttggaaggtgatttgatgagagggtggcacccttatgaatgggattggtgcccttatcaaagaggcccagaggtgaggatgtggcaagatggcaccttctacgagccatacctctcaccagggaccaaatctgctggtatcttgaacttgggctttccagcctccagaactgtgagacagagaaataagccaggcgaagtggctcacccctgtaatcccagcattttgggaggctgaggtgggtgatgacttgaggtcaggagttctagactagcctggccaacatggtgaaaccccatctctactaaaaatacaaaaaattagtcaggcatggtggcgcatgcttgtaatcccagctactcgtgaggctgaggcaggagaatcccttgaacccgggaggaagaggctgcagtgggccaagatcgtgccattgcactccagcctggggggaaaaagatagaaataaatctcttgtttgtaagcccctccgctttaaggtgttttgttttcgcacctgaacggactaagggactaagggaccaggaatcatccaggcctcagcttcccaaagcactccccactccccagttcctacagggccccgaccccagccccagcacccgtcattcacattttttacttacaaaggaaactttgaaatggacacttcctgttactttcattcaatgaaaaaaatccccagcacttgaaggcaatcatacagaaagcctgcaacaacgcagagagtagactcttcaggtggtggtgcctgctttaggctggctctccccgggtcaaaagggacgtgataaagtgttaaagtgatgttaaggatcaagtgggcaccaactgcgactcccctcccaccccccgacttaacagatgtcaaggaaaacagaaggaagccgtttctctccaggacatgcagtgtctgctggagttttggaaaaatgctttctctcgagtcgctgcggggagctcccaggctggacccccccgcacaggtcctccgccatcgccctcccagcccccgcccccaatccccctcccagacccggtccccgcccccatcccccgccagacgtcgtccccacccccatccccctccaagccccgcccgggagggcgtcctgcccgctcattgggcctcccgccggctcatttgcatggtccgaggggggcggggctgacgtcgcgctgggaatgccctggccgagacactgaggcagggtagagagcgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctgggtgcgtctggggacgctgcccgggctccacgtgcggagtgggtgccccctaggccggggagcgggggatccccaggggtcgcggggccctggaggagcgggcatcggacgcggacacggcggggtgcatcccgagggccccctccgaggcagatgcttcctgcgggggcgctgttcctgggcccgggaagggggcgttggaaccccgagcggtccgggccgaagcctgggactctcgtgcgtccccacccctacccccatcaggcgcccgtgcatgaagggagaccctcacctccggactgagagtcggagcgtctcggagcgacggggagtagggagcgggacccggggcggagggtagtgctggcccctgcggactccgggtcccctgtgtcctctcgggaggggctggacgggctgagctgccgaggggccgatttgccctgggccggacaaagagtggggctttggccggtcccccacggtgggctccttccctctggggattgagggactcaagacaccccgcgcctgcgcttttcttttctttttttctttttttttttttgagacggagtttcgctcagtcgcccaggctggagtgcagtggcgtgatctcaactcactgcaagctccacctcccaggttcacgccattctcctgcctcagcctcccgagtagctgggactacaggcgccagccaccaagcccggctaattttttgtattttttagtagagacggggtttcaccgtgttagccaggatggtctcgatctcctgacctcgtgatctgcccacctcggcctcccagaatgctggggttacaggcgtgagccactgctccctgctgcctacgctctctgggtcgcagcccagccttctgggggctgggtagcctcccagaagggcaaccctgggcatcctccagggcaggctaactggagtctagtggggaggggtaccttgaaagaggaaagttgtttcctcctcctcctcctcctccagtgtttgggacccttcctgggggctggagtgcatccctggacaccccccaatcccatcctcttctctagtttccactgacctaggcccaccctcccctctccggctcagtactcctggaaatgagattccgtacatttgaatcttgtcctaatgaaatatttgtccatgtgggtacctgtgtgtgtgtggtgggggtgcagacggagggtttgtttctcactagctggaactactggggtgtggtatgcttcctgggaatttgtgtgccacagtcctggaggcgaggagggggttgtgagccagtaggcaggggctggggcaagtagcattgtgaagctattgacacccagacgtccccaggcaggagattatgcccccattagcccccttttatctgggcttccttaacaatggactctttgccctgcctgccagagccagcagggagtgactgttcagtggtgaggaagcgggcagaggaagccctgccattgggtaggagcagtgggcagcccctgggctgactgggaggtggggattagggattagacagtcctggctgtctgccttcccctaagccagggggagaggagcaaagggcacgaaatgtggcctccaggaggattagaccgccacatgatcatttgcacaccctggggtttagcaacaataaaagtcagcttttttgtatcccaaggtggcctgtggacacccacatggacaaatgtttacactgggacagaattcaaatgcagaggtcccaggagcctaaagtacactcactctggtatagaaaggattccttactgggcagaggacaggtgcagcctggggctttcccaggcaggacacagggaggctcaggaaccaccaagtccctggaaggtggatctggaggcgttggcaggagccactccctgggttccagggctccaggttcctgctttaaccccctgtctcacagagggctgtgcacttgggggctgctgagcatgtcccagaggctgcatcctggacacagcacctcagtgcatctgagctgaggctaacttggcaggagggacaggcagaacctgccagccacgtgcaattccacccctctggccactcagggaaggagagctgtgagtcaagatcagatttgggtcaggacaggctggggcctgcctgtccctgtgcatcccaagatttatggctggccaggggttgggctgggaggggtggtcttgcatgccaggagagtgcagatcagcctgagaggccaggccagtaagtgaggtcagatctcctgcacctgatagcattaaggccatctacaccaaagctctaatgctgatatgttcctggcctctatgtggggcatggaggtggggcatggaggtgaggcctgctcgcctgggcttctggaagtgggagactcattcctgtggctgaggcctacagcagtgctgtgtggtaggaatacactggaagccatgatgtcattgtgcattttctagaagccacattgaataaagtaaaagacacaggtagaattaatttcattgagcccaatatatccaaaataatatcattttcacatctattcaatataaaaatttactaatgagatatttcatactaagccactgaaatccagtttgtatcttacacatctcagttttgacgagccacatttcaagggcgtgatagccacatgtggctcccatagtagacagtactggtctagagaaatgttggtggcatccttgctgtctggtttctggccttgccaaaagtattaccatcccagtgtggtacattctttcatgtatttgtctcctgtccccagagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgtaagcagtttgcttatctggacgttgtcaagttagaaaagctgttttgggatgggtgtggtggctcatgcctgtcatcccggcactttgggaggccgaagcgggtgggttgcttgagcccaggagctcgagaccaacatgatgaaacccagtctctacaaaaattacagaaaaattagctaggcatggtgttgtgggcccatagtcccagctactagggaggctgaggcaggagaattgcttgagcctgggaggtggaggttgcagtaagtcatgatcatgccactgtactccagcccgggtgacagtgagatgctgtctggaaaaaaaaaaaaaagaaagactgttttgttttggaagcaacacaggcagttgtaggccccctgtgccagagtgacataaactctgtacacctccagtgatttggtccatgtttgtaaaccctgaatgttccagggcagtttcttttcttcactttttatctcttttttttgggtgggggggcggggtacagagtcttgctctgtctcccaggctggagtgcagtggcgcaatctcaacctcccgaggagctgggactacaggcacaggccatcacaccttgctaatgtttgtactttttgtagagacggggttttgccctgttgcccaggctggtcccaaactcctgcacccaagtaatctgcccacctctgcctggcagttacaatttcaaataattcctccctttccttcaacacttggctcatgaccgtccagtccaaggaacctgtcctgcaggtgtgcctctcccgagcttcctctatgcatcttccataatgaagatgccttctcactggaaaccctacaagggtgggaacgtgccttatttgcctgtatcctcagggtctagcagagagaagataatttgtaataccaaaacaccattaaattcagctgatgctttcataagcgctccttggaggaaggactccatttacttgacagatctgtgcaagacagcagcctggcgcgtctaacctgcagccagttgcatcctctgtttaaccttgtttgcggaagctttctctaaacagccagcacttgtctgttcccacatgggtccgttctcccagtgaatcaccgtggtgcctgctgactgctctgtagcacagtgcttcgcaaagtgtgatcctgggaccagcagagcagcagctcctttgagcttattggaatggcagaccctcaggtcccacctctgacctgctgcatgggaattctggggagggacgcagaatctctggttccacaggctctccggtgatgctaatgaataccggcatttgaacagcaccgatctagcccctttcagtccatgagccaacaacccttggtcctgtctgtggtgacccagtgtgactctcatggggagcaaggagaggaagttgaagttcactgacagggttgttaaggggattatgcaatagatgagacccatgggcctgaagtccgagggtgtatgttagttccccgttcttttgacccatggattaacctactctgtgcaaagggcattttcaagtttgttgccctgctcacttggagaaagcttatgaaggatcaggaaaattaaaagggtgctctcgcctataacttctctctcctttgctttcacaggccttggtatgttcctgcttcatccccttctacagtggccttatccctccttccttcagaggcgtggtaagtcggctttctctgctagcgctgagtcctgggggcctctgaagtgtgctcacacatctcctgcctgcagggcactggtgtcgggcacctcagggtctgtcccatggtggagccccatgcctcactgcctttcagacagagtagccacagctggccctatttccaggctacccgggcagcaaaacttactgcatgtgtaattaattatttggctatctgtaaggtaaactggctggttcacttaatctgcaccttaagcatcagatagcttctcagtgatctagttaaactatatgatgttggccaggcgcggtggctcatgtctgtaatcccagcactttgggagcctgaagcaggcagatcacttgaggtcaggagttcgagaccagcctggccaacagtgtgaaactctgtctctcctaaaaatacaaaaattagctgggcatggtggtgtgcacctgtaatcccagctgctcgggaggctgaggcaggagaattgcttgaacttgggaggcggaagttgcagtgagccaagatcgcaccactgcactccatcctgggtgacagagcgagactctatctcaaaaagaaaaaaaaaaaaaaggtaaataaagtatatgacactgaagaatctgttacccctggaaggtggagctttactcttagggggaactataacagtcatatatatatatttttttcttttctttttttttttttttgagatggagtctggctctgtcgcccaggctggagtgcagtggtgcaatctcggctcactgcaacctccacctcacaggttcaggcaattctcctgcctcaacctcccgagtagctgggattacaggtgcctgccgtcacgccaagctaatttttgtatttttagtagagacagggtttcatcatattggccaggctggtctccaactcctgacctcaggtgatccgcccgccttggcctcccaaagtgctgagattacaggcgtgagccatggtgcccggccaacaatcacatgtgttgtaaacaacaacaaaaatctgtcagcctggtctaacctagatttgtgctttgttttgttttgccactttgtgatgcacaggaggaagtttaggctgtaaaatactagccttttagggtaatttttgaactcacaagagcagcagcggaacctttgatgcaatcctgtatgtagcaccagcagagccacgtggcagagggactcgcattaggagcctcccattacagactacgtgctcctgtgcgttatcttatagggtccccacaaccaaggggagatgtgattattcatcctgtgtggctgtggggaacttgagagtcatacttgcccaaagagcacggccagcgagcttgcacccaggtcactctctgctcctctgtcagaacagggcatgtcttggttcactgcagggcggctcttctcattctctgtagtttggggtccaggatagtggtccacggagccactggagtgcccagctactgagtgaccaaagcatattttggatttccgacattgccacagcatggttgggcatcagcaggaccccaaccccttgttatgctggtggctttatgtggttatttgatcttccccagaactcagcaggagtgcacccagcagcaccgtagtgatgctctctggctccccagtgcacggttctggctttccttcctggtcgagagtttcaagccctctgggtcctactctgtccttttcagcccatagctttgttcaaaagctgctggcagtgttcagatttggctgagttcagtgaatatgtgcattggctgatttctgagccatgccagggggatggagaagccgaagcaggagtgtttgttctgcaggctctggagtaggcattgggtctgtgccggctcacttgctagtcttgcatccttccccaaccccctctggggatgtctggccacatcagaagacagtttgggttgtcagaactgggggagtaccaggccgaggtgggtggatcatgaggtcaggagatcgagaccatcctggctaacacagtgaaacctcatctctactaaacatacgaaaaaaattagctgggcgtggtggcgggcgcctgtagtcccagctactcgggaggctgaggcaggagaatggtgtgaacccggggggcggagcttgcagtgagctgagatcctgccactgcactccagcctgggcaacaaagcgagactccgtctcacaaacaaaacaaaacaaaacaaaacaaaatctgggggagtgccactggcatctgatgtatagaggcccgagatgctgtgtcatcacccgttgagtgcgctcataggcatcttcctgacaattagaacccattattcttcaaattcaatgcaagcaaattcaaagcattactgtgtacataccgcatgctaatcaattgcaccactggagctcctaaattcaaaacattactataaaaaagttcaaaatgcatggaaaagttgtacatggcaggagaatatttgggcttctgactaccccttgaatgaagatgatccaccagccgccttcctccttggtcttcactccagattcctagcatttcattctgtgtctctttatgcagtgaggtttttgtttgttttttgagacagagtctcactgtatcacctaggcctggagtgcagtggcgcgatctcagctcactgcaaccctcggctcctgggtttaagcgattctcctgcctcagcctcccgagcagctgagattacaagcacacatccccatgcccagctaatttttgtatttttagcagagacagggtttcaccatgttgcccaggctggtctcgaactcctggcctcaagtgatccatgtgcctcagccttccaaagtgctgggattacaggcgtgagccaccatgcccagctcctagtgaggtttttgatgccttgctacatctgccctagaaattgtgtgactacgattttggaaatgttgctgtgtaaacttgtgatcatttctggactccaggcaagaatcttgatggctaaggtgtggctgaacatgtctgattctctcctggacctgttttaggccaaactctgctctgaaattcctccgtgtggaagggcgggctggggagagcctcccagctggaatcttttggatgcctttctctgtgggtatctgatggctggctctgatggctggctgtgatggctgtggctggaaatcattgttgacatgagtttcacagatgcaggctctgtccaaattgtagcaaaagctgcctgccccagccgagctatgggcaataaggtggtttaaggatatagatgaaggaaaactcacccttagaataatttatccaaaatgctgctgtgttgtgggttagaggacattttctgaggtcccaggttcattgtttcatttaagtctcaaaagtccctccaggtgttggttctaattgtcaaagcatggggggagatgggctcatgggttaaaggtcttatcccagatttctgtatcctccttgcaagcagcaaaggggtctggatttgaatccatgaccatgtttctcctttgggtttccatcacactctgtccccgtgcactgagcaccctttagttcatatgacccccttaggcatgttacatgggcactcctataggtgcccatctggccctaggacttggccaacacaacatggactccagtttccatctgcctctttgccaggcacttttgtgcagtgcacacactgtacaacagtagacggcaaccctgagagccagagtagagcctgtcctagcaccggaatgctcggtaaggatttgtcgcaggagtgattccaaagccaatgtcctccctccatatcagcctgtttgtggctctgagaagctctgcccacatgtgaaagcttgttaagcacttaagcactaacccagagcttcagacagtgccagtcctttttccccttctttaaaagcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgagttggtggtgagggggcaggtgttctggggtgcagctcttctttgcctccctgattgccaggagctaccagttactgtctgcacaatcaaacagaaatagacctgtccttgatggttaacggaaataaaaggcgcttgtcccagaagctcaggtgaggcaccaccctgattatgggaatcacctgggaacatatacccagacctaaaactcagatccacttcccaggctgtggttatatagtcaggggggtgcagtatgggtattaggattttttattttttagttataaagatttttttttggtttgtttttgagacagggtcttgctctgccgcttaggctggagtgcagtggtgcaatcatagctcactgaagcctcagactcctgggttcaagcagtcctcccacctcagcctcctaaggagctgggacccacaggcatgcagcaccacacctggctaatttttaaaaattttgtggagtgttgcccaggctggtctcacactcctggcctcaagcgatcctcccaccccagcctcccaatgtgttgggattacaggcatgagccattgtacccagccactaagatgattcttatttggaaacacggtcaagaacaactgcgttcggtagtttaaccttttttgattgtggtggttttagtatgccttaccactctaccatagtaagaaatttgcagaccatgtacaccaacctttggtgctcctggggagaaagaaagaaggctatgcaatgcaatgcatgctcacagtccaagggagagggaaagctgtctaacaggattggttttcccgtgtgctttataagcagatgagtagaggagacagctcttattgtcctagtggcaattgggataggctgcaaagtttgttagggtggaggcttattccgggaccaagggagcccaaagaaacaagctcctgccaggcgcggtggctcacgcctgtaatcccagcactttgggaggctgaggcaggtggatcacctgaggtcaggagtttgagaccagcctggccaacatggtgaaaccccgtctccatgaaaaatacaaaaattacccgggcatggtggcgggcacctgtaatcccagctactagggaggctgaggcaggaaaatggcttgaacctcggaagtggaggtggccgttagccgagatcacgccactgcactccagcctgggcaacagagcaagactctgccttaaaaaaaaaaaaaaaaaaaagaaaagtaaaaggaaaaaaaagaggctctggcctgctggggtgcctgcaaagtctccgtggaagggtgacattcaagccgagacctccagggaactgtctcctgggagcacagagccctttgctcagcccccaggtggctcagtgcccccagccagcagactcagagcttgcatgattctttggtgctctctgcggtcttccaatgatgctgaaataaatggtgcttggtgtctccctgctgtagtccccttgcttgctttgctcacaggtgctgggagagatatgccttcgaggatatttggatgcattcaggttcttggaagagaagggtatgtatgggctgggaggatcagccatgcccttttgacaagcatttactagcggtcttggtaaagacttgagatttgccttagttctaacacttagtgcccaacgccttccttgtgttgctcaacctactcatgagcccaggagataggaaatctccgtcccattgtacagatggggaaacagaattttggaaaggagagccaagcagcacacacccctccctgaggggcagagccgagatttgaactgggatgtcatgactccagggccctctccctccccagggtccccttatctgaaggcggtttttctttccagctcgacctcttgtgacccttagtttaacaagggccgaagttaaagagtttctgcgcctggaccccaaatgaagcaatcagatttctcatctccagtcaggtgtgggtccaagcccactagacaagtttgctcttcccagagcacatttctgccttcaagtcatcctggcttgtcagggctgggggagttctgctgtagaaatattagagtggaaggaaaaagatgtgttgggagctatttttctttaatactaaaagttggttgatgaatttgtcgttggccaagaccaaggagactgcatttttaaggacatatgtgtatttatctgctcagaaaatgttcattgctgtgtgctagggatactgcagtgaacacagaggtgtgacccttgccagccttgtgagagaagtgagcagataagtaagcagaagggtgatgctgtgtcgatgggaaagtacaggtgccaatgagaaggcacaggtgtcaaggagaagacacaggatgctggaggctcatgcaggatggatctccaaggcccaggggaagaagggcctctcggaggacgtgaatccacattaagactttggggataagtaggagcgccttaggcatggggacccatggatgcgaggcctgtaggacacagacaggatggcatgaaggcctgtgcaactggaggggtggggatggggacactaagagatggctggaagtgtgggggtggggacactaagagatgactggagaagagggggtcaggagtggtgaaaaatgggagaggagggcaggctgggccttttggatacagggggattgcatcctgcagtggtagggagccactgagggctgctgcagtaggagtgaggggatcagaggagagctttggaagccccctggatgcgggacaggaagcgagataccagtgtctaggaggccagtgaggcagccacaggctccaccaggatcagggctgcgagggtcatgaggaggaaaccaatttgaaggagtccaggggaataggacttggaaatgaccgatgggacatttgggaagaggaagacagaagagcgcagtcccggcttctggctttagcagttgggcaaggggagatggggagatgtgcccatgggttgagggttgaggacattaggagggagccggtatggcaggaagagctggtgtgccagagatgctggaagcagcatctgcctgagaacagatacctggcaatattcctaagggaaagtgacatctcggagggtgaggagggcatctgatagggcctggaaagagccggggcaagcatgaatgtgaggttatcttggggggcaaggctcaggcgttgaggagcagcccctggtctcttcagcctgaagttggaagccagagttgggccaggtgcagctgtggttgtctgaagtccccctcccccagcccagtgtgccaatgctgtaagagcaagggccgctcactggtgctggtggctgagtcccagcacccaggacagggcctggcacatactggtgcccaatcctcccttctgggtgcttcttccaaggccttgtgatggaagtgagtaccctcttcgacatcagacccagcttcaaatcctggctctgctatgtattggctgcgtggctttagacaagtcttttaaccttgctgtgcttctgatttctcagctgaaaaatggagatgatgatagtggtttctgtaaggccttatggtgaagcacctagctcagggcctggaaggcaggtgtaaccagtggttcagttgttataaaccaacactaaccctcgcctttgcacctcatgaaaccagatatgtagatggagcccacaaagctagcaggagccaagctcacgtgtgtcctgctttaaagccccatacccctttctccgggtgacaaacacctgtgctcgttctcttcccttcccctcttccccttgcatttggctaataacaggccagctgcctgcctccctgcagtttggtagatgggtgggtaacgaccaccactcccacgttcgcctgatgggcttgttttccgtgcccttcacaggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacaggtacccactcctcggggtgagcacgggcagcaccttgttttctttcttgtgcattatggaggaagatggtactgccacatgggagcgatagggtgaggcaaccatgacaggtggttgggaacatctccttccatgtgtacagcctgggctgctgccatcactcccagcacagcccccaacccccccaatcctggaaccttgccaagtctcccttcccatggggtcatgaccaggaggaaaacaaactccagctgagccccttggggttccccatataggctcctgcctgtggcagctgggccctctgtacccctttccaactctgtctccctaacatggcacctgagctcctgccatcctggatttcatggaccccaaggatgggggtcctgcatctgggacttggcctattactcggagctccttttcagccgcctccctccacctgtccacccacctcaaggctcctttcttgagacctctcctaatttctcccttcccctaaacccacaattttgaacctccatcgaatggtgctgtattttataatgtcatcaaatatcaaatggagacagtgctatggtccaaatgattgtgtaccccccagaatttgtcttttgaaatcctaacccccaacatgatggtcttaggaggtggggcctttgggaggagattaggtcatgaggaaagggctgtcatgaatgggattggtgcccttattaaacagacccaagagaggtcccttgtcccttctactgtgtgaggactcagaaggtggtgtctatgaagaagcaggccctcaccagacaccaacatgtctgctgccccttgatctgggaccttgcagcctctagaactctgaaaaatcgatgtttgttgttttataagccactcagttggtggcattttgttagagtagcctgaacacggactaagtcaaacagaagaacccacaaaccagctacagagttgggcatttggagaaattcaaaaatgagtcagacataactccttattcttgaggtgccctaagagatgggacacagcagctgcccaggtgcattagtttgttctcacattgctataaagaaatacctgagactgggtaactcataaagaaagaggttgaattggctcacagttgcacaggctggacaggaagcatggtgctggcatctgctcagcttctggggaggcctcaggaaacttacaatcatggcagaaggtgaacgggaagcatgcacatcccatgactggagcaggagtgagagagagagggaaatagagggaaggtgccatacacttttaaacaaccagatctcatgagaacacattcactatcaagagaacagcaccagtggggaaatccgcccccatgatccaatcacctcccatcaggctccgcctccaacactgggaattacaatttgacatgagatgtgggcagggacacagatccaaaccatatgaccagattaatacgatttgaggcatcacgaggtcattaaagagagggaataaaagactggggctccaggaagaaggctctggaatccagcagagggtcaaggaccagcttgtaaagctggtggtgcctgagaagtacctaggagaacatagatgctgtgacgtttgatgtagctgttttttgttttgtgttttggtttttgagacagagtctcactctgttgcccaggctggagtgtgcagtggcgtgatcttggctcactggagcctccatctcccaggttcaaatgatcctcatgcctcagcctcctgagttgctgggattacaggtgcacaccaccacgcctggctaatttttgtgttttcagtagagacagggtttcaccatgttggccaggctggtcttgaactcctgacctcaagtgatccaacaacttcagcctcccaaagtgctgggatgacaggcatgagccaccatgcccagcctgatgtagctgtttctgtgcacattatttgctgtggggtatattcagatttcttaatacaagatgattctttgcctcatgacttacacaccattttctatttaatttcagctatgatattggaaatggacatgtcttttcaaggaaaataaaagcaggctttctggaatggcgacttccaaacatatttgtcaatttaaaggagctgggagtggggaccctatgctccgtaagcactctcttagctgttcttggctgtgctccccgcttcagcttcacactgcccttgctgtgaagggagcagcctgggccgggcgcggtggcttacacctgtaatcctagcactttgggaggccgaggtgggtggatcacctgaggtcaggagttcaagaccagcctggccaacatggtgaaactccatctctactaaaaatacaaaaaattagctgggcatggtggcaggtgcctgtaatcccagctacttgggaggctgaggcagaagaatcgcttgaacccaggaggcggaggttgcagtgagccgagattgcgccattgcactccagcctgggggcaacaagagcaaaactctgtctggaaaaaaaagaaaggagcagcttggcaaaccccaccttgtcgcttttgtgagtgcctctgaccctttggctgccaggacgggcgtattttatggaaatgctaagcaccaacagagtaaagtggtttggtttttcacagtggtgggagataatagctccaaattgtctttttcagcactgagtgaagaaatgaaagacaaaggtggatacatgagcaagatttgcaacttgctacccattaggataatgtcttatgtaatgctgccctgtaccctgcctgtggaatctgccattgcgattgtccagaggtgagcattttaggtggctccgtgtcttcctcacagggttgatatgaggatgaaacaagatgatagatcatggtggcatgtagtctgggacctggattgtcgtgccacagatcacagctcacagtctatgtgcaatgcccctgaatgttgcccacctgtcctcaagccacacatgcacctgtaactcagtgcaagcccagaaactccccgtggggactcctagagctgtcagtggcctcacatagcagctggtccagtctcttgtgattgcccaaggaaactgaggcctggagagcttggggtcactgctctgaggccatagagatgcctagtagaagggccaggcctagaagcaggatccttgctgcccctctgagctgtttccatttaaaatcacatgaaggccggcgccgtggctcacggctgtaatcccagcattttgggaggccaaggtgggtggatcatgtgaggtcaggagtttgagaccagcttggccaacatggtgaaatgccatctgtactaaaaatacaaaaattagtggagcatggtggcacgtgcctgtactcccagctacttggaaggctggggcagaagaatcgcttgagcctgggaggcagaggttgtagtgagccaagattgtaccactgcactccagcctgggtgacaggagagaaaccctatctcaaaataaaatgaaaggtaatgaaatgaataaaataataaatcaagtcacggccgggcacggtggctcacacctgtaatcccagcgctttgggaggccgaggtgggtggataatgaggtcaggagttcaagaccagcctggccaacatggtgaaaccatgtctctactaaaaatacaaaaattagctgggcatggtggtgcatgcctgtaatcccagctactccggaggctaaggcaggagaattgcttgaagcaggacctaggaggcggaggttggttgcagtgagccgagatcatgccactgcactctagcctgggctacagagcgaaactccgactcaaaaaaaaaaaaaaaaaaaaatcaaatcacatgaaagtagaacatagggaattccatctttcgttctaggcatagtttgttaatatgattcagagccagcagttaggagaacacagtgtgactctcctagaacttcttgattgggcttcctctgattgggtttcctctgattgggcttcctctgaaagtgggggggatggggggtggggagcagaatggtcagagcttggctcagcagtcagactgctcttcttcaaatcctggctgcattgcttactacagctgtgtgactccagatgactgaatccacctctctgtgctgcagcttcccgtctagagagatcacctggagcagagggtggtcaggagactcaatctggttactgactcacagtgcaggagtactcatcccatagtaagcatccagctagagatgttgatttctattttcaggtaataatgatgatcgtaaaattagagacagataaaaggtatgggcattaggccagggcactgcaatttctaagctgtgtgacctcaggcaagttactcgacttctctgagcctcagcggtttcatccgcaatatatggataggaaaaccgacctcagtgggttgtctgacagtggagggcacttgattaaaaaaaaaaaaattaccctggtctgaatattaccctggactgaaagaaaaatattgagctaatacaggcatcaggaatggggctgcagggagtccagggaagggagaacgaagagcctgaaggtgtgaggaggtgcgagtgctgatctgtctgctacaaagaggctgctgagcctcctgtggatgtggccctggacttggcagtttaatacctgagctgttaaaataacctcagatgctgtgttctttaaggggtaggattcagattcctgctgaaatgcttctgaaagggagggaatgagccagcccatccccagttgctttttaagatcattgggaagttctggtcttgccatttgtccctggaccactcttaggtcctcctgccccacttccatctgggtgtgtgccctgggctgtccaccacacagctacatcctgccatcttccctcctggagccactgtgccatgcatggatctgtagcttcatttttcttggcttttccctggtttttctggagcagagtctctagtaaactcccaaggaagaaaacgtttgactttatgtgtgttgggaaacgtgctttttttctattacatctcagtgataggttggccatgtctagaattgcaggttgaaaatcatttcctctcagtatattggttagtgagaagcctgggactgagacagtcacattctcacttctttgcaggtgagtgctcttaggactgtctttttatcccttatactctgaaatgtcatatgtcttggtgtaagtccttatttcagttattgagctggacaagtactggagaccccttcagtcaaagccttctgtcattctccagctctaggaaattatcttctattgttatttctgttattccttcccttccattttcttttttctttttttttttttttttttgagacagggtcttactctggtgcccaggctggaatgcagtgacctgatcatggtacactgcagcctgaacctcccagactcaagtgatcctcccacctcaacctcctaagtagctgggactgcaagcacacatcaccacacccaacaaatattttttaaaaattttgtaagatgggatcttactatgttgcccagactttttcttcctcttcctggggctcttattaggaagatgtttgacttcctgggttggattcctgtctccgtgtctgactttctctctttgtcatatttttcatcactcgttgtctttttgcgtctgctctgacagatttcctcaaattttgtcttctagtcctatcctacagtttttactttcagcaaatataatttaatctccaagagtactctcttgttcttttttcttagcattctgttcttgttttatggatgtaacattctcttggaatatttgctgtcctctagatcatcccttctccatttcttcttgggctagtttttctgtttcttcatctttctcttttatgctacttattctgggcgtgttcttggtgggttttttcccatatagcaacagaggacttggagctcagggagaaaagggtaggtgcatcacctggcagagctcccagacagtgacaggcaggctgcgggaaggatgtctacttggcggtgctaccgctttcctagaaaccctttccctggagctggttgaactgttgggttttgccctggtggtgaacgctggctccccgtgctctgcctgtttcatcaccagccccctccccttctgcctggggtccagtaatctgttgaaatatatatcttgctcattggtgagctcctgctccttcctcgttgctcttgcagatttatcacttctcgtaaggctgcgcttgtacttcgggattttctctgtgccacactgggaaacatagggtggttgcatgctgcagtcctgagcacttatttcactcacatctttacacgaagatttggtgggtgtttactttgtttttagtaagttagtctgtcatgtcctttgatcctttttttttgttttttgagatggagtctctctgtgtcctccaggctggagtgcaatgtcgcgatctcagctcactgcaacctccacctcctgggctcaagagattctcctgcttcagtctcctgagtagctgggattacaggcatgtgccaccacacctggctaatttttgtatttttagtagaggtggggtttggcatgttggccagcctggtctcaaactcctgacctcctgacctgcctgccttggcctcccaaagtgctgggattacaggtgtgagccaccacacctggccctgattaatcttttaatgcccagtctctccttcaaaagccggctcctttctctccctcgccttcctagattccttctccactccccaggatcagcctcctcctccccaccccaccactgccggggggatgtctgtggtcaggcatttatcagagaccctgaggtgggggtcctttatgtgtctgggggatggagagtctagaggaggtagcgttcagacctctccatggtgcctctgctgggctcacatgtgaccaagcacagcaaaccatgaggcaggggatggtcttgaccatgagagcccttgcagcagctgccatgggcctcagctcctctccaagctgggaagagccctgaaaagccaaggtgtttttttttccctctttatttcagtgtaagtcccttgagctttcttgaaccagaagtgggctcattttgctttagagatttcaggtgggcttgtccttgtcctagcatcccagatccaccttctgggaagtcatcagattggaggtgatgttggcagcttttgtaaacaaagggtagtgttgtaagctgttgtgtctgcctatgtgtgtgtttgtgtacttggtctcatctctgcagactggtgacatggcttccagatatgcccgacgatgtcctgtggttgcagtgggtgacctcacaggtgttcactcgagtgctgatgtgtctgctccccgcctccaggtaaatactttggctgtgggtgtgtgggccggacgggcacctctctcatctgatgaggcctcacacgacattctagaaacagctggctgaacaccaagcaaggagcttgcccttgggtgtggggaccctgtctcatgggaggcagctgagtcagtcagaggtcctggcacacctgctgagagctgccacccaggccaacctgaaccggagcctgggaagacttcccgtcggatgagtctctttgagtgcagcattgatggtggaagagcagagaggccccagataagcagggaaaggtgcttcagacagagtggctgggatgaggactggggagtgtcagatagcgctggcgtgtctgagcgaaggagctctggcacccatggcacaggaaggaggtgggaccctggaggggcagggctagcagagctcctcggagcgtgtggctaggtgcctggtaatgcaagccccctgtcctccaccctctgttgtactgagtcacagtctccggggtgaagcctagcagtctgcgttgacaggccccaggggatgccgctacttcctgaattctgaattctggaaactgagccggagttcagggcctggctcccattaccagggttgggcgttatcctgaaaatcataggccttggtttcctcacttggctaacaggggtgatccccatcccctcaatgggtttccgtgagctcctgagagcccgtagcatggtacttggcacatgctgggcatcaggaggtatggcctctcttgctattgttgttattggtagacacagaaggatttaaaagtaggggaatgcaaagatccgatttgctagggaagagggcagtagtggccaagtagagggtggatcctgggccctggctggcagcaggcagcaaggggggctgccagggcccaggcagggacgatctgtagaccgagaggcttcctaaggctcttggacaggaggaggtgtcggttccaagcctaaggagtggggcagccctggtgactggtggtcagtggtgccaggcggtgggtggtaggacaccctggcaggcaagtaggtttgtgtgggggaaactgataggcccctccagggattcgttggtggacaacacctgtgatgtccagtgggaggtgtccaggtagctgggagggccacaggcttggaagacctaggtggtgacatcagcccagcactgagggctagaagaagctgtgtctctggctgtgacggcaccctagagtgtgtgtggtgccctctactggccggcaatgtgggtccaccgtagctcagactgcacactgcagcagcgggaacggcctctaagccaacttcctccatgtgtttcaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagccttaaaaaaaaaaaaatcggttgggtgcagtggcacacggctgtaatcccagcactttgggaggccaaggttggcagatcacctgaggtcaggagttcaagaccagtctggccaacatagcaaaaccctgtctctactaaaaatacaaaaattatctgggcatggtggtgcatgcctgtaatcccagctattcggaaggctgaggcaggagaatcacttgaacccaggaggcggaggttgcggtgagctgagattgcaccatttcattccagcctgggcaacatgagtgaaagtctgactcaaaaaaaaaaaatttaaaaaacaaaataatctagtgtgcagggcattcacctcagccccccaggcaggagccaagcacagcaggagcttccgcctcctctccactggagcacacaacttgaacctggcttattttctgcagggaccagccccacatggtcagtgagtttctccccatgtgtggcgatgagagagtgtagaaataaagacacaagacaaagagataaaagaaaagacagctgggcccgggggaccactaccaccaatgcgcggagaccggtagtggccctgaatgtctggctgcgctgttatttattggatacaaagcaaaaggggcagggtaaagagtgtgactcatctccaatgataggtaaggtcacgtgggtcacgtgtccactggacaggggtcccttccctgcctggcagctgaggcagagagagagaggagacaaagagaaagacagcttacgccattatttctgcatatcagagacttttagtactttcaataatttactactgctatctagaaggcagagccaggtgtacaggatggaacatgaaggtggactaggagcgtgaccactgaagcacagcatcacagggagacagttaggcctccagataactgtgggcgagcctgactgatgccaggccctccacaagaggtggaagagcagagtcttctctaaactcccccagggaaagggagactctctttcctcgtctgctaagtagcgggtgttgttccttgacactttttgctaccgctagaccacggtccgcctggcaatgggcgtcttcccagatgttggtgtcaccgctagaccaaggagccctctgatggccctgtccaggcataacagaaggctcgcactcctgtcttctggtcacttctcgctacgtcccctcagctcctatctctgtatggcctggtttttcctaggttatgattgtagagtgaggattattataatattggaataaagagtaattgctacaaataatgattaatgatattcatatataatcatatctaagatctatatctggtataactattcttgttttatattttattatactggaacagctcgtgtcctcagtctcttgcctcagcacctgggtggtttgccacccacaattttccacatgcttctggtctgcgttctttttttctccttgcatcttctcattctctgatcaccccaacctctctctgtcttccctactctgccagccttgatggagacaagcccttgagaccagaactcaccttttccccaggtgtgataatctattagcaaggtcagtgtaatatgactgacggtgaaacgtgtatttttttctatttatgcatttgagtacagtacgtacaagaaaaataatggtgtgctcaaactgttaaatgttggaaaagaaagatacaacccttacccatattgtgtaagtgccctggagtagaagaaccagcaagctcagacaaagcacttgactgagaagacagaccctttaaaggaaacgggttctagggacaaactctacgtgggcctgttctcttgataagaccgtgaactctttgagaaaagaggctacttgtgaaaataatgagcccccttcggggcaggagttccggggtttgaacctgccttcttacatcttgagggctaagtgagttcccaaggcctctgttcagtggttgctccgtcagtgagctcaggtctggtgagtggcagggtcttccacccccaaccccaccgggtgtcagagcaagacactgtcccccatggagctggaatggggtggaggagcccacatctggcacccacgtggcctccttgtgacggacccacccttgcaatgttggaaaggaaagttacaagtttcttttcccaagtttcccagtaggctttgttctgttagcttcacgccttcggtcattagcagacataaataaactttaaccattgttttttatcttttttttttttttttgagatggagtttcactcttgttgcccaggctggagtacaatggcacaatctcggctcaccacaacctccgcctcccgggttcaaacgtttctcctgcctcagcctcccaagtagatgccaagcctcccaagtagatacaggcatgtgccaccacgcccagctaattttttgtatttttagtagagatggggtttctccaggttggtcaggctgatctcgaattcccgacctcagatgatccacccgcctcggtctcccaaagtgttgggattccaggtgtgagccaccacgcccggccaacagtattttctaataaccagtatatttccatatacatgtgtacatgggtattgtgattgttatcaggaaaaaatatattaaatggctgataggagaccatgggacgtattttctttctgcttttaaaaattattcaggccgggtgcggtggctcatgcctgtaatcccagccctttgggaggccaacgtggacagatcacctgaggtcaggagttcgggacaagcctggccaacatggtgaaaccctgtctctactaaaaacacaaaaattagccaggtgtggtggcaggcgcctgtaatcccaattagtgggttggctgaggcaggagaatcacttgaacccaggaggtggtggttgcagtgagctgagatcgtgccactgcaccccagcccgggtgacagagtgagactccatctcaaaaaaataaaaaataaaaaataaaaaataaattattcaatcttctttatttttattgttttaatgactaaagttatttttagtagaaacagggtcttgctatgttacccacgctagtcttgaactcattggcttaagcagtcttcctgccttagcctcccaaagtgctgggattacaggcatgcctggctcctccaccttcttaaaataagcatttgttttataattttttccaagtgtgtatcaagataaggaaatcaggaagtgtaatattcttatagaaatggccaaggcctcccccttcacctgtgcctcagatgctacccaatcccgccttctctgtccctccagaaggcaccctttgcttaggcctccctctcttcctgaaccacctctggaagtttcttattggcctatgaatgcattcttatttcttcttatcaaataaagccttccctttaatttatggcacatttatgctttggaatccactctcaggaataatcagtatgtagcatattacacgtcaggcggcaacattctttttttttttttttttttttgaggcagggtcccgctctgtcatccaggctagagtacggtggtgcaatcatagttcactgcagcctcaacttcctggggtcaagccatcttcccacctcagtctcccaagtagctggaaccacatgtgtgcactaccacacccagctagtttttttttgtagagacagggccttgccttgttgcccaggctggtctccaagtcctgggctcaagtgatgctcctgccttggccttccaaagtgctaggattacaggtgtgagctaccatgcctggtccaacattcttcatttggtaaatggctaaacttagtgcagagtatgagcctgattttgtttaaaaaaaaaatgtgtgtgggtgtgtatatgtatctttgagtgtatataaaaagactgaaagagggctgggcacggtggctcatgcctgtaatcccagcactttgggaggctgagggaggcggatcacttaagtcaggagtttgagaccagactgggtgaaaccccatctctactaaaaaatgcaaaaattagctgggcatggtggcgggcacctgtaatcccagcaaccagggaggctgaggcaggagaatcacttgaacccaggaggtggaggttgcagtgacccgagatgcagtgagactccacctcaaaaaaaaaaaaaaaaaaaaaaaaaaagactaaggatatatatcaaaacccttatggcagactgttatttgtaattgtattttatttgtcgtgcttatatgtgttgcccaagtttctatggtgaacggtatgcatcactttcagcatgagaaaataactcctaataaacgtgattcttaaaggactctccctgtgtacatcctttccaaggagccccgatgtaccctgcttccctcacagccaagctcctctaggacagtcctccctagggggttacggccttgctcctttgatggccctgccacagcccaggggctctttcctgggcactgtggaccagaccctcctagaatccctctccttcctcaaccccagactctcaggcaccccatctctatcctggacaccgagcttgtcaccaaggcccagctgcagctgctgtgctgagtgtgcacagccacctctgggaaggctgtgctccccctgaggacctgggtcctcagtctctgactctggggggattcggggccacctacccaccttctggctctactcaacaccagcagcccctcaacatcaagccctccagctcacagcacctcagctctgcagctccaaagccaccccagcctggaactggggactcagggagactccctcgctccaactccagctggtcccagcactgcctctgctgtcatctctggaatctggtctcttgtccccagcccactgctgccagggtcagccctggtactttctacctgggttgctgcaacagcttccctctgggggccgcctgttttcagccttgggcccatctgccctccacgttgctgctgggcaagctgaggccccaccacacccccagcaccttcctgtgcctccaggccttccctgccagtacaccccctccagcccctgaagccctgtcagcagctcagagcctttccccggagtcaccctgcttcccgaggggacccacgttgctgctgggcaagctgaggccccgccacaaccccagcgccttcctctgcctccagactccctcctagcacaccctctccagctcctgcagtcccaccagcagctcagagcctcttccctgagtcaccctgcttcccgaggggacctccacccagggaatgcccttccaggctcctctcactgattcaccttcactcctcagaggtcaggtgctggctgatagaggcaatgaggtgagcccctcacctcagggcgctcagcccctgggaaggtgatgaaaaagggcaccccaaggggtgtcccaaactaataggtggatgactcccgtgggaagatccagtctcccctccccatggccaattaaatttggggtgtttttctttacttttttgaggcaaggtcttgctttgttgctcaggctggagtgcagtggcacaatcacagctcactgcagccttgacctcctgggctcaagcaatcctcctccttcagcttcctgaggaggttttaattaaataaattaaaagctaattaaaaacatttttttttttttttggtagagatggggtctcactgttttccaggctgatcttgaactcctggactcaagcaatcctcttgcttcagcctctggagtagctgggactacaggtgcttgctactatgcccagctattttttttattttttatttttattttgtagtgatggggtgtgtttcctaggctgatcttgaactcctgggctcaagcaatcctcctgcctcggcctcccaaagtgctgagattataggtatgagccattgcactcagcctggattgtattaaggaaactagcttagtcactaaactgcacttctacttctgattggacaagattggcactttgtaaaccaggttttctgggatgctggtttccttgaagatgtttctagctggtattatcatataggataggctgagttatgctgcattaacaagcaaaacccaaaaggtttatttctcattcgtgctgtatgtccagcaaatgtggctgggcttcattttaagtcgtcctcactcccagacctagcctgtaggtgccccaccattgggggatcgctgattgccatggcatggttagagaaatatggttcttaaaggttctcatccagagtggcacatctctcttctgctcacaatgtgttgaccaaagcaagtcacatggatgggagcagggagatgcaatcctctcttgtgcccaaaagaggagcacaagaaatactgttgaatagcattgccagaggccacagaaaaatctccaaaaccaaaaatctctatgacccaataaattcaagaaatactgggttaaagagaaggaaaccagtatatgcactgtgagacttctcagtctttattatatacattgtaaccatcacagacagcctttaatatatacatcttaactatcacagacagccttcagtatattatatacattataaccatcacagacagccttcaatatattatatacattataaccatcacagacagcctttaatatatacattataactatcacagcctttaatatattatgtacattgtaactatcacagcctttaatatattatatacattgtaactaccaccgcctttaatatagtatacccattgtaactatcacagcctttaatatacacattgttaactaccacagacagcctttaatatattatatacattgtaactatcacagatagcctttaatatatacattgtaactatcgcagacagccctttgtttgagggtaaagcatgtgactttgttttcctcctgttccctctgccatcctccagggcctcacacacaataggctctcaaatattgcaaccatcttgcactctgcagggcaacctcatggcttatcctttacaagtggcatactcagaagaataacgaggaagaaagtgtaagaaaagagggctgtttgggcaaagaaaatctggttgattcgatgtttacagagtgccaggccccatgcttgatctcatgaggtcctcagaccttatgccttaggctatcatttccattttacagataaggaattgaggctcagggaactgagagtgagggaagggcaggggcctggctggtgctgggttctgtctctttccaggcaagaagggacatgtggacttgaatatttagctcagggatgttggaggcctagggtgctgctatttaaatgg

SEQ ID NO: 2 - DNA, 16 bp, Artificial Sequence
  Description of Artificial Sequence Synthetic oligonucleotide
ctttattcaatgtggc

SEQ ID NO: 3 - DNA, 16 bp, Artificial Sequence
  Description of Artificial Sequence Synthetic oligonucleotide
tttttagtagtcaagg

SEQ ID NO: 4 - DNA, 16 bp, Artificial Sequence
  Description of Artificial Sequence Synthetic oligonucleotide
cgtttttagtagtcaa

SEQ ID NO: 5 - DNA, 16 bp, Artificial Sequence
  Description of Artificial Sequence Synthetic oligonucleotide
ttggatatattgggct

SEQ ID NO: 6 - DNA, 16 bp, Artificial Sequence
  Description of Artificial Sequence Synthetic oligonucleotide
ctagtaaatgcttgtc

SEQ ID NO: 7 - DNA, 16 bp, Artificial Sequence
  Description of Artificial Sequence Synthetic oligonucleotide
agaaattgcagtgccc

SEQ ID NO: 8 - DNA, 16 bp, Artificial Sequence
  Description of Artificial Sequence Synthetic oligonucleotide
cttagaaattgcagtg

SEQ ID NO: 9 - DNA, 16 bp, Artificial Sequence
  Description of Artificial Sequence Synthetic oligonucleotide
taatattcagaccagg

SEQ ID NO: 10 - DNA, 16 bp, Artificial Sequence
  Description of Artificial Sequence Synthetic oligonucleotide
gtaatattcagaccag

SEQ ID NO: 11 - protein, 29 aa, Homo sapiens
HSQGTFTSDYSKYLDSRRAQDFVQWLMNT

SEQ ID NO: 12 - protein, 30 aa, Homo sapiens
HAEGTFTSDVSSYLEGQAAKEFIAWLVKGR

SEQ ID NO: 13 - protein, 31 aa, Homo sapiens
HAEGTFTSDVSSYLEGQAAKEFIAWLVKGRG

SEQ ID NO: 14 - protein, 30 aa, Artificial Sequence
  Description of Artificial Sequence Synthetic polypeptide
  chimeric GLP-1/glucagon peptide
HSQGTFTSDYSKKLDSERARDFVAWLVAGG

SEQ ID NO: 15 - protein, 30 aa, Artificial Sequence
  Description of Artificial Sequence Synthetic polypeptide
  chimeric GLP-1/glucagon peptide
HSQGTFTSDKSEYLDSERARDFVAWLEAGG

SEQ ID NO: 16 - protein, 30 aa, Artificial Sequence
  Description of Artificial Sequence Synthetic polypeptide
  chimeric GLP-1/glucagon peptide
HSQGTFTSDKSRYLDSRSARDFVAWLEAGG

SEQ ID NO: 17 - protein, 30 aa, Artificial Sequence; identical to SEQ ID NO: 15
  Description of Artificial Sequence Synthetic polypeptide
  chimeric GLP-1/glucagon peptide

SEQ ID NO: 18 - protein, 30 aa, Artificial Sequence
  Description of Artificial Sequence Synthetic polypeptide
  chimeric GLP-1/glucagon peptide
HSQGTFTSDKSSYLDSRSARDFVAWLEAGG

SEQ ID NO: 19 - protein, 30 aa, Artificial Sequence
  Description of Artificial Sequence Synthetic polypeptide
  chimeric GLP-1/glucagon peptide
HSQGTFTSDKSSYLDSRRARDFVAWLEAGG

SEQ ID NO: 20 - protein, 30 aa, Artificial Sequence
  Description of Artificial Sequence Synthetic polypeptide
  chimeric GLP-1/glucagon peptide
HSQGTFTSDKSKYLEGQAAKEFIAWLEKGR

SEQ ID NO: 21 - protein, 30 aa, Artificial Sequence
  Description of Artificial Sequence Synthetic polypeptide
  chimeric GLP-1/glucagon peptide
HGQGTFTSDYSKYLDSKRAQDFVQWLVAGG

SEQ ID NO: 22 - protein, 30 aa, Artificial Sequence; identical to SEQ ID NO: 14
  Description of Artificial Sequence Synthetic polypeptide
  G931
  MOD_RES (13)..(13): Lys(gE-palm)

SEQ ID NO: 23 - protein, 30 aa, Artificial Sequence; identical to SEQ ID NO: 20
  Description of Artificial Sequence Synthetic polypeptide
  G934
  MOD_RES (10)..(10): Lys(gE-palm)

SEQ ID NO: 24 - protein, 30 aa, Artificial Sequence; identical to SEQ ID NO: 18
  Description of Artificial Sequence Synthetic polypeptide
  G973
  MOD_RES (10)..(10): Lys(gE-palm)

SEQ ID NO: 25 - protein, 30 aa, Artificial Sequence
  Description of Artificial Sequence Synthetic polypeptide
  chimeric GLP-1/glucagon peptide
  MOD_RES (2)..(2): Gly or Ser
  MOD_RES (10)..(10): Tyr or Lys
  MOD_RES (12)..(12): Lys, Glu, Arg, or Ser
  MOD_RES (13)..(13): Lys or Tyr
  MOD_RES (15)..(15): Asp or Glu
  MOD_RES (16)..(16): Ser or Gly
  MOD_RES (17)..(17): Glu, Arg, Gln, or Lys
  MOD_RES (18)..(18): Arg, Ser, or Ala
  MOD_RES (20)..(20): Arg, Lys, or Gln
  MOD_RES (21)..(21): Asp or Glu
  MOD_RES (23)..(23): Val or Ile
  MOD_RES (24)..(24): Ala or Gln
  MOD_RES (27)..(27): Glu or Val
  MOD_RES (28)..(28): Ala or Lys
  MOD_RES (30)..(30): Gly or Arg
HXQGTFTSDXSXXLXXXXAXXFXXWLXXGX

SEQ ID NO: 26 - protein, 30 aa, Artificial Sequence
  Description of Artificial Sequence Synthetic polypeptide
  chimeric GLP-1/glucagon peptide
  MOD_RES (10)..(10): Tyr or Lys
  MOD_RES (12)..(12): Lys, Glu, Arg, or Ser
  MOD_RES (13)..(13): Lys or Tyr
  MOD_RES (17)..(17): Glu, Arg, Gln, or Lys
  MOD_RES (18)..(18): Arg, Ser, or Ala
  MOD_RES (27)..(27): Glu or Val
HSQGTFTSDXSXXLDSXXARDFVAWLXAGG

SEQ ID NO: 27 - protein, 30 aa, Artificial Sequence
  Description of Artificial Sequence Synthetic polypeptide
  chimeric GLP-1/glucagon peptide
  MOD_RES (10)..(10): Tyr or Lys
  MOD_RES (12)..(12): Lys, Glu, Arg, or Ser
  MOD_RES (13)..(13): Lys or Tyr
  MOD_RES (27)..(27): Glu or Val
HSQGTFTSDXSXXLDSERARDFVAWLXAGG

SEQ ID NO: 28 - protein, 30 aa, Artificial Sequence
  Description of Artificial Sequence Synthetic polypeptide
  chimeric GLP-1/glucagon peptide
  MOD_RES (10)..(10): Tyr or Lys
  MOD_RES (12)..(12): Lys, Glu, Arg, or Ser
  MOD_RES (13)..(13): Lys or Tyr
  MOD_RES (27)..(27): Glu or Val
HSQGTFTSDXSXXLDSRSARDFVAWLXAGG

SEQ ID NO: 29 - protein, 30 aa, Artificial Sequence; identical to SEQ ID NO: 14
  Description of Artificial Sequence Synthetic polypeptide
  chimeric GLP-1/glucagon peptide

SEQ ID NO: 30 - protein, 30 aa, Artificial Sequence
  Description of Artificial Sequence Synthetic polypeptide
  chimeric GLP-1/glucagon peptide
HSQGTFTSDYSKKLDSRSARDFVAWLVAGG

SEQ ID NO: 31 - protein, 30 aa, Artificial Sequence
  Description of Artificial Sequence Synthetic polypeptide
  chimeric GLP-1/glucagon peptide
  MOD_RES (12)..(12): Lys, Glu, or Arg
HSQGTFTSDKSXYLDSERARDFVAWLEAGG

SEQ ID NO: 32 - protein, 30 aa, Artificial Sequence
  Description of Artificial Sequence Synthetic polypeptide
  chimeric GLP-1/glucagon peptide
  MOD_RES (12)..(12): Lys, Glu, Arg, or Ser
HSQGTFTSDKSXYLDSRSARDFVAWLEAGG

SEQ ID NO: 33 - protein, 30 aa, Artificial Sequence; identical to SEQ ID NO: 15
  Description of Artificial Sequence Synthetic polypeptide
  chimeric GLP-1/glucagon peptide

SEQ ID NO: 34 - protein, 30 aa, Artificial Sequence
  Description of Artificial Sequence Synthetic polypeptide
  chimeric GLP-1/glucagon peptide
HSQGTFTSDKSEYLDSRSARDFVAWLEAGG

SEQ ID NO: 35 - protein, 30 aa, Artificial Sequence
  Description of Artificial Sequence Synthetic polypeptide
  chimeric GLP-1/glucagon peptide
HSQGTFTSDKSRYLDSERARDFVAWLEAGG

SEQ ID NO: 36 - protein, 30 aa, Artificial Sequence; identical to SEQ ID NO: 16
  Description of Artificial Sequence Synthetic polypeptide
  chimeric GLP-1/glucagon peptide

SEQ ID NO: 37 - DNA, 16 bp, Artificial Sequence
  Description of Artificial Sequence Synthetic oligonucleotide
tatttttggtgtatcc

SEQ ID NO: 38 - DNA, 16 bp, Artificial Sequence
  Description of Artificial Sequence Synthetic oligonucleotide
ggccaatacgccgtca

SEQ ID NO: 39 - DNA, 24 bp, Artificial Sequence
  Description of Artificial Sequence Synthetic primer
gaggaatcagatgaggatatggga

SEQ ID NO: 40 - DNA, 20 bp, Artificial Sequence
  Description of Artificial Sequence Synthetic primer
aagcaggctgacttggttgc

SEQ ID NO: 41 - DNA, 26 bp, Artificial Sequence
  Description of Artificial Sequence Synthetic probe
tcggtctcttcgactaatcccgccaa